(19) United States
(12) Patent Application Publication
(10) Pub. No.: US 2023/0248414 A1
(43) Pub. Date: Aug. 10, 2023

(54) SELECTIVE MODULATION OF INTRACELLULAR EFFECTS OF CELLS USING PULSED ELECTRIC FIELDS

(71) Applicant: Virginia Tech Intellectual Properties Inc., Blacksburg, VA (US)

(72) Inventors: Michael B. Sano, Cary, NC (US); Christopher B. Arena, Blacksburg, VA (US); Scott S. Verbridge, Blacksburg, VA (US); Rafael V. Davalos, Blacksburg, VA (US)

(21) Appl. No.: 18/123,719

(22) Filed: Mar. 20, 2023

Related U.S. Application Data
(63) Continuation of application No. 16/443,351, filed on Jun. 17, 2019, now Pat. No. 11,638,603.
Application No. 16/443,351 is a continuation of application No. 13/332,133, filed on Dec. 20, 2011, now Pat. No. 10,448,989.
Application No. 13/332,133 is a continuation-in-part of application No. 12/757,901, filed on Apr. 9, 2010, now Pat. No. 8,926,606.
(60) Provisional application No. 61/424,872, filed on Dec. 20, 2010.
(60) Provisional application No. 61/167,997, filed on Apr. 9, 2009.
(60) Provisional application No. 61/285,618, filed on Dec. 11, 2009.

(51) Int. Cl. A61B 18/14; A61N 1/32

(52) U.S. Cl. CPC A61B 18/14; A61N 1/327; A61B 2018/00761

(57) ABSTRACT

The present invention relates to the field of biomedical engineering and medical treatment of diseases and disorders. Methods, devices, and systems for in vivo treatment of cell proliferative disorders are provided. In embodiments, the methods comprise the delivery of high-frequency bursts of bipolar pulses to achieve the desired modality of cell death. More specifically, embodiments of the invention relate to a device and method for destroying aberrant cells, including tumor tissues, using high-frequency, bipolar electrical pulses having a burst width on the order of microseconds and duration of single polarity on the microsecond to nanosecond scale. In embodiments, the methods rely on conventional electroporation with adjuvant drugs or irreversible electroporation to cause cell death in treated tumors. The invention can be used to treat solid tumors, such as brain tumors.

### CROSS-REFERENCE TO RELATED APPLICATIONS

The present application is a Continuation of and claims priority to and the benefit of the filing date of U.S. Pat. Application No. 13/332,133 filed Dec. 20, 2011, which published as U.S. Pat. Application Publication No. 20120109122 on May 3, 2012. The ‘133 application relies on and claims priority to and the benefit of the filing date of U.S. Provisional Pat. Application No. 61/424,872 filed Dec. 20, 2010. The ‘133 application is a Continuation-In-Part (CIP) application of U.S. Pat. Application No. 12/757,901, filed Apr. 9, 2010 (patented as U.S. Pat. No. 8,926,606 on Jan. 6, 2015), which relies on and claims priority to and the benefit of the filing date of U.S. Provisional Pat. Application Nos. 61/167,997, filed Apr. 9, 2009, and 61/285,618 filed Dec. 11, 2009. The ‘133 application is also related to International Patent Application No. PCT/US11/66239, filed Dec. 20, 2011, which published as WO 2012/088149 on Jun. 28, 2012 and which claims priority to and the benefit of the filing date of U.S. Provisional Pat. Application No. 61/424,872 filed Dec. 20, 2010. The entire disclosures of all of these patent applications are hereby incorporated herein by reference.

### STATEMENT OF GOVERNMENT INTEREST

This invention was made with government support under Contract No. CBET-0933335 awarded by National Science Foundation. The government has certain rights in the invention.

### BACKGROUND OF THE INVENTION

### Field of the Invention

The present invention relates to the field of biomedical engineering and medical treatment of diseases and disorders. More specifically, embodiments of the invention relate to a device and method for destroying aberrant cells, including tumor tissues, using high-frequency, bipolar electrical pulses having a burst width on the order of microseconds and duration of single polarity on the microsecond to nanosecond scale.

### Description of Related Art

Electroporation based therapies typically involve delivering multiple, unipolar pulses with a short duration (~100 µs) through electrodes inserted directly into, or adjacent to, the target tissue. See Nuccitelli, R., X. Chen, A.G. Pakhomov, W.H. Baldwin, S. Sheikh, J.L. Pomicter, W. Ren, C. Osgood, R.J. Swanson, J.F. Kolb, S.J. Beebe, and K.H. Schoenbach, A new pulsed electric field therapy for melanoma disrupts the tumor’s blood supply and causes complete remission without recurrence. Int J Cancer, 2009. 125(2): p. 438-45; Davalos, R.V., L.M. Mir, and B. Rubinsky, Tissue ablation with irreversible electroporation. Ann Biomed Eng, 2005. 33(2): p. 223-31 (“Davalos 2005”); Pavselj, N., V. Preat, and D. Miklavcic, A numerical model of skin electroporation as a method to enhance gene transfection in skin. 11th Mediterranean Conference on Medical and Biological Engineering and Computing 2007, Vols 1 and 2, 2007. 16(1-2): p. 597-601 (“Pavselj 2007”); and Pavselj, N., Z. Bregar, D. Cukjati, D. Batiuskaite, L.M. Mir, and D. Miklavcic, The course of tissue permeabilization studied on a mathematical model of a subcutaneous tumor in small animals. Ieee Transactions on Biomedical Engineering, 2005. 52(8): p. 1373-1381.

The extent of electroporation is attributed to the induced buildup of charge across the plasma membrane, or transmembrane potential (TMP). See Abidor, I.G., V.B. Arakelyan, L.V. Chernomordik, Y.A. Chizmadzhev, V.F. Pastushenko, and M.R. Tarasevich, Electric Breakdown of Bilayer Lipid-Membranes .1. Main Experimental Facts and Their Qualitative Discussion. Bioelectrochemistry and Bioenergetics, 1979. 6(1): p. 37-52; Benz, R., F. Beckers, and U. Zimmermann, Reversible electrical breakdown of lipid bilayer membranes: a charge-pulse relaxation study. J Membr Biol, 1979. 48(2): p. 181-204; Neumann, E. and K. Rosenheck, Permeability changes induced by electric impulses in vesicular membranes. J Membr Biol, 1972. 10(3): p. 279-90; Teissie, J. and T.Y. Tsong, Electric-Field Induced Transient Pores in Phospholipid-Bilayer Vesicles. Biochemistry, 1981. 20(6): p. 1548-1554; Zimmermann, U., G. Pilwat, and F. Riemann, Dielectric breakdown of cell membranes. Biophys J, 1974. 14(11): p. 881-99; and Kinosita, K. and T.Y. Tsong, Formation and Resealing of Pores of Controlled Sizes in Human Erythrocyte-Membrane. Nature, 1977. 268(5619): p. 438-441.

Once the TMP reaches a critical voltage, it is thought that permeabilizing defects, or pores, form in the plasma membrane in attempt to limit further TMP rise. Pore formation can either be reversible to allow for the introduction of foreign particles into viable cells, or irreversible to promote cell death through a loss of homeostasis. Known devices and methods of performing electroporation clinically involve several drawbacks, including painful muscle contractions, unpredictable treatment outcomes, and a high potential for thermal damage in low passive conductivity tissues.

IRE performed with unipolar pulses causes intense muscle contractions. Therefore, clinical applications of IRE require the administration of general anesthesia and neuroparalytic agents in order to eliminate the discomfort caused by muscle contractions seen during each pulse. See Talele, S. and P. Gaynor, Non-linear time domain model of electropermeabilization: Response of a single cell to an arbitrary applied electric field. Journal of Electrostatics, 2007. 65(12): p. 775-784. Receiving paralytic agents is undesirable for patients, and may deter them from seeking an electroporation based therapy. Further, in some cases, even with an adequate neuromuscular blockade, muscle contractions are still visible (see Pavselj 2007), and questions remain as to what constitutes an appropriate dosage. Muscle contractions may affect the location of implanted needle electrodes, which can invalidate treatment planning algorithms. Additionally, in treatments near vital structures, displacement of the implanted electrodes may cause unavoidable collateral damage.

The time course of the pulsed electric field and dielectric properties of the tissue control the TMP development and the extent to which the transient defects form and reseal within the membrane. Knowledge of these two components can be used to predict treatment outcomes. However, predictions are complicated in heterogeneous tissues, or organs with multiple types of parenchymal tissue. There is often an intricate and unknown geometrical arrangement between tissues of low and high electrical conductivity, and the conductivity can change in real-time due to the phenomenon of electroporation, the extent of which is highly unpredictable without prior knowledge.

Low conductivity tissues, such as epithelial layers, often contain a dense packing of cells with reduced extracellular current pathways. As such, the resistance of the extracellular space is increased. Additionally, when pulses much longer than the charging time of the plasma membrane (~1 µs) are applied (see T.R., A.T. Esser, Z. Vasilkoski, K.C. Smith, and J.C. Weaver, Microdosimetry for conventional and supra-electroporation in cells with organelles. Biochem Biophys Res Commun, 2006. 341(4): p. 1266-76, “Gowrishankar 2006”), the current is confined to the extracellular space prior to the onset of electroporation, as shown in FIGS. 1A-B. As shown, when the pulse duration (t_{d}) is much less than the plasma membrane time constant (τₚₘ), current flows through both intracellular and extracellular spaces (FIG. 1A). In the case that t_{d} is much greater than τₚₘ, current flow is restricted to the narrower extracellular spaces (FIG. 1B). Consequently, there is a large voltage drop across tissues with low conductivity, which increases the potential for deleterious Joule heating effects, such as thermal damage.

### SUMMARY OF THE INVENTION

The present invention provides advancements over conventional tissue electroporation by utilizing high-frequency, bipolar pulses. Pulsing protocols according to embodiments of the invention involve bursts of bipolar pulses with a burst width on the order of microseconds and duration of single polarity on the microsecond to nanosecond scale, as shown in FIG. 2. The total burst width of the high-frequency pulses (~100-1000 ns duration of single polarity) is on the order of hundreds of microseconds, the time delay in between bursts is on the order of seconds, and the total number of bursts can be adjusted.

It is possible for the electric field to penetrate tissue heterogeneities when high-frequency electric fields are employed, because capacitive coupling is enhanced allowing current to flow through both extracellular and intracellular spaces. See Gowrishankar, T.R. and J. C. Weaver, An approach to electrical modeling of single and multiple cells. Proceedings of the National Academy of Sciences of the United States of America, 2003. 100(6): p. 3203-3208; and Ivorra, A., ed. Tissue Electroporation as a Bioelectric Phenomenon: Basic Concepts. Irreversible Electroporation, ed. B. Rubinsky. 2010, Springer Berlin Heidelberg. 23-61. In this case, all cells present in the organ, regardless of their packing and morphology, experience a macroscopically homogeneous electric field distribution. See Esser, A.T., K.C. Smith, T.R. Gowrishankar, and J.C. Weaver, Towards Solid Tumor Treatment by Nanosecond Pulsed Electric Fields. Technology in Cancer Research & Treatment, 2009. 8(4): p. 289-306. This results in more predictable and uniform treatment outcomes without the electric energy being preferentially deposited into regions of tissue with a lower passive conductivity. As a result, Joule heating is also more uniformly distributed throughout the tissue, which mitigates the potential for thermal damage in regions with a low passive conductivity.

Enhanced capacitive coupling also limits the change in tissue electrical conductivity due to electroporation. Therefore, prior knowledge of how the conductivity of a tissue is modulated in response to electroporation is not required to accurately predict the electric field distribution. As a result, simplified algorithms can be implemented for treatment planning.

High-frequency, bipolar waveforms are also included in embodiments of the invention for mitigating or completely eliminating muscle contractions during electroporation based therapies. It is well known in the field of functional electrical stimulation that the threshold for nerve stimulation increases as the center frequency of bipolar waveforms increases. Further, muscle twitch forces are reduced as frequency increases. The present invention demonstrates that a range of frequencies exist where non-thermal tissue ablation can be achieved without causing nerve excitation or muscle contraction. In the context of this specification, it is noted that the term ablation is used to indicate destruction of cells, but not necessarily destruction of the supportive stroma.

Clinically, this translates to performing IRE without the requirement of paralytic agents (or a reduction in the amount of paralytic agents administered) in all procedures, and without the further requirement of general anesthesia in minimally invasive procedures. Additionally, other complications caused by IRE with unipolar electric pulses are alleviated, including electrode displacement and pain associated with intense muscle contractions.

Examples of heterogeneous systems include, but are not limited to, tumors surrounded by or containing any type of epithelial layer, such as a skin fold geometry, or systems comprised of multiple tissue types including, brain, bone, breast, pancreatic, kidney, or lung. In this specification, an epithelial layer is defined as a dense packing of cells that restrict the flow of materials (e.g., electrical current) resulting in a low passive electrical conductivity.

The present invention applies to all electroporation based therapies. Recently, electroporation has been utilized in vivo as a means to destroy cancer cells within tissues in both reversible and irreversible modalities. Reversible electroporation is being studied to facilitate the delivery of anticancer drugs and DNA into cancer cells through the plasma membrane in the form of electrochemotherapy (ECT) and electrogenetherapy (EGT), respectively. Irreversible electroporation (IRE) promotes cell death resulting in the development of a tissue lesion. It is an independent means to ablate substantial volumes of targeted tissue without the use of harmful adjuvant chemicals if used prior to the onset of thermal injury. See Davalos 2005. By not relying on thermal processes, IRE has been shown to spare the extracellular matrix and architecture of nerves and blood vessels.

More specifically, the present invention provides new devices and methods for the treatment of diseases and disorders, such as hemic and solid neoplasias, which improves conventional clinical practice associated with electroporating target tissues.

Included in embodiments of the invention is a method of treating a subject suffering from a neoplasia comprising: implanting at least one device for emitting electric pulses into or adjacent a neoplastic site within the body of a subject; and delivering one or more electric pulse to the neoplastic site, such that amplitude and duration of the pulse are in the range of about 1500 V/cm to 2500 V/cm for 10 µs or less which is capable of inducing irreversible electroporation. Methods of the invention also include non-invasive methods of treating a subject comprising non-invasively placing at least one device for emitting electric pulses around a region of the body containing a neoplastic site within; and delivering one or more electric pulse, such that amplitude and duration of the pulse are in the range of about 1500 V/cm to 2500 V/cm for 10 µs or less which is capable of inducing irreversible electroporation.

According to embodiments of the invention, such methods can employ multiple pulses administered in a pulse burst having a duration of less than 10 ms.

Such methods can employ one or more pulses or a plurality of pulses in a pulsing protocol, wherein the amplitude of the pulse is in the range of about 500 V/cm to 1500 V/cm. Amplitude in the context of this specification refers to the voltage-distance ratio of a pulse, such as for 1500 V/cm the voltage is 750 V over a distance of 0.5 cm.

Such methods can have a pulse duration in the range of about 2 MHz (250 ns) to about 500 kHz (1 µs). For example, the pulse duration can be about 1 MHz (500 ns). In preferred embodiments, the duration of each pulse is in the range of about 100 to 10,000 ns.

Any number of probes or electrodes can be used invasively, semi-invasively, or non-invasively according to embodiment of the invention. In preferred embodiments, two or more electrically conductive regions are used within a single device for emitting the electrical pulses. Similarly, in any of the methods according to the invention, two or more devices can be used to deliver multiple electric pulses at different positions within, on, or near a body.

Custom treatment area shapes can be created through varying electrode activation patterns in combination with any of the embodiments of the invention.

The methods can also employ delivery of a bipolar burst of pulses. In embodiments, a bipolar burst of pulses can be delivered with multiple pulses in a single phase before a polarity switch. Even further, total burst width of any pulse protocol according to the invention can be between 1 µs and 10,000 µs. In preferred embodiments, the methods can have a duration of single polarity within a bipolar burst of between about 100 ns and 100,000 ns.

The shape of the electric pulses delivered using methods of the invention can be square, ramp, sinusoidal, exponential, or trapezoidal.

In preferred embodiments, two or more electric pulse bursts can be administered with a delay between bursts. In preferred embodiments, a delay between bursts can be on the order of seconds. For example, in bipolar protocols a selected positive voltage (+V) can be applied for a selected period of time (e.g., 50 µs), then a zero voltage applied for a selected period of time (e.g., 75 µs), then a negative voltage (-V) can be applied (e.g., 50 µs). The voltage can be applied in any number of individual pulses, as a pulse or pulse burst.

Also included in embodiments of the invention is a method of delivering electric pulses such that amplitude and duration of single polarity are selected to be capable of administering electroporation to electrically excitable tissue without stimulation of the tissue.

Further included is a method of delivering electric pulses such that amplitude and duration of single polarity are selected to be capable of administering electroporation to electrically excitable tissue with reduced stimulation of the tissue as compared with higher amplitude and longer duration pulse protocols. Preferably tissue stimulation that is avoided or prevented refers to a muscle contraction.

In embodiments, the neoplastic site, region of the body, or electrically excitable tissue can be nerve tissue, muscle, or an organ containing nerves and/or muscle tissue.

Any embodiment of the invention can employ applying electric pulses having an amplitude and duration in the range of about 1500 V/cm to 2500 V/cm for 10 ms or less which is capable of inducing irreversible electroporation.

Method embodiments of the invention can be used to build up the transmembrane potential of a tissue to a critical value (~1 V) by delivering trains of less than 1 µs bipolar pulses. For example, multiple monopolar pulses can be delivered at a pulse duration of about 5 MHz prior to a polarity switch, then delivered at a pulse duration of about 5 MHz after polarity switch.

Methods of the invention may or may not employ administering of a drug designed to induce a neural blockade. The methods can include administration of general, local, or no anesthesia for treatment of tissues with electroporation based therapies. In preferred embodiments, no neural blockade is required for treatment of tissues with electroporation based therapies, or lower dosages of a neural blockade can be used in embodiments of the invention to achieve the same results as using higher doses with lower frequency pulsing protocols.

The pulses of any method of the invention can be delivered on a short enough timescale to flow through epithelial cells but are long enough to induce electroporation in underlying cells. In specific embodiments, a frequency of 500 kHz or 1 MHz or 250 kHz is used to treat underlying fat cells in a layer of fat disposed under the epidermis.

Methods according to the invention can be modified to provide for administering non-thermal IRE, IRE, and/or reversible electroporation.

Treatment planning according to embodiments of the invention can result in more predictable outcomes in homogeneous and heterogeneous tissues than compared with lower frequency pulsing protocols.

Any one or more of the methods, devices, or systems, or parts thereof, can be combined with other methods, devices, systems, or parts thereof mentioned in this specification to obtain additional embodiments within the scope of this invention.

Devices and systems for implementing any one or more of the above mentioned methods are also within the scope of the invention.

### BRIEF DESCRIPTION OF THE DRAWINGS

The accompanying drawings illustrate certain aspects of some of the embodiments of the present invention, and should not be used to limit the invention. Together with the written description the drawings serve to explain certain principles of the invention.

FIGS. 1A-B are schematic illustrations showing electrical current pathways through epithelial layers and bulk tissue prior to the onset of electroporation.

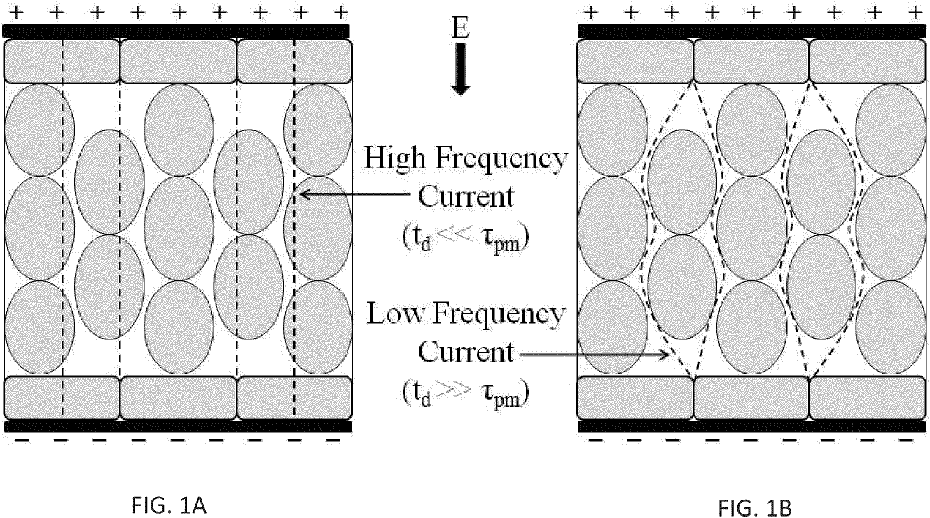

FIG. 2 is a schematic diagram of a representative pulsing protocol for electroporation based therapy according to embodiments of the present invention.

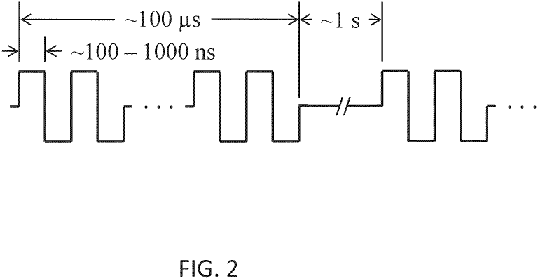

FIGS. 3A-D are graphs showing dielectric properties (σ and εᵣ) as a function of frequency for skin and fat.

FIGS. 4A-B are graphs showing respectively 2 MHz AC burst with a width of 4 µs, and a DC pulse with a duration of 4 µs of equal amplitude (FIG. 4A); and magnitude spectrum of the AC burst (thick) and DC pulse (thin) (FIG. 4B).

FIG. 5 is a graph showing frequency (ƒ) response of the TMP at the cell pole (θ = 0) for rectangular bipolar pulses (-) and sinusoidal waveforms (- -). The box illustrates the frequency window implemented in the FEM.

FIG. 6 is a graph of the strength-duration curve for unipolar electric pulses expressed as electric field strength in tissue. Adapted from Rogers, W.R., J.H. Merritt, J.A. Comeaux, C.T. Kuhnel, D.F. Moreland, D.G. Teltschik, J.H. Lucas, and M.R. Murphy, Strength-duration curve for an electrically excitable tissue extended down to near 1 nanosecond. Ieee Transactions on Plasma Science, 2004. 32(4): p. 1587-1599 (“Reilly 2004”).

FIG. 7 is a strength-duration graph comparing unipolar to bipolar rectangular and sine waveforms expressed as phase charge and current. Adapted from Reilly 2004.

FIGS. 8A-B are schematic diagrams showing meshed geometry of the FEM with boundary settings (FIG. 8A) and the geometry with dimensions (FIG. 8B).

FIGS. 9A-D are schematic diagrams showing the electric field, norm (V/cm) contours predicted by the FEM at the end of a 2 µs burst with an amplitude of 2600 V and a frequency of 250 kHz (FIG. 9A), 500 kHz (FIG. 9B), 1 MHz (FIG. 9C), and 2 MHz (FIG. 9D).

FIGS. 10A-B are graphs showing temperature changes predicted by the FEM at the center of the skin (FIG. 10A) and fat (FIG. 10B) for various frequencies of 250 kHz (- -), 500 kHz (- - -), 1 MHz (· · ·), and 2 MHz (-).

FIGS. 11A-B are graphs of TMP predicted by the FEM at the center of the skin (FIG. 11A) and fat (FIG. 11B) for frequencies of 250 kHz (- -), 500 kHz (- - -), 1 MHz (· · ·), and 2 MHz (-).

FIG. 12 is a diagram of a system for implementing high-frequency, bipolar pulses for tissue electroporation.

FIGS. 13A-B are graphs showing output of the arbitrary function generator prior to signal amplification by the high voltage MOSFET positive and negative polarity switches.

FIGS. 14A-B are micrographs showing in vitro experimental results on electroporation with high-frequency bipolar, pulses using a trypan blue dye exclusion assay.

FIGS. 15A-C are waveforms of IRE with unipolar pulses and high-frequency IRE with the corresponding TMP development across the plasma membrane (Φₚₘ) for a 1500 V/cm unipolar pulse (FIG. 15A) and a 1500 V/cm bipolar burst without a delay (FIG. 15B) and with a delay (FIG. 15C).

FIG. 16 is a graph comparing time above the critical threshold (Φ_{cr}) for IRE at various center frequencies.

FIGS. 17A-C are waveforms of IRE with unipolar pulses and high-frequency IRE with the corresponding TMP development across the plasma membrane (Φₚₘ) for a 1500 V/cm unipolar pulse (FIG. 17A), a 1500 V/cm bipolar burst without a delay and with a shortened negative phase (FIG. 17B), and a 1500 V/cm bipolar burst with a delay and with a shortened, lower amplitude negative phase (FIG. 17C).

FIG. 18 is a chart showing an exemplary output from an in vivo treatment of the brain with high-frequency, bipolar pulses, where the snapshot is taken within a single burst.

FIGS. 19A-B are photographs showing histological sections of liver tissue treated with high-frequency IRE (FIG. 19A) and conventional IRE with unipolar (FIG. 19B), with cross sections of tissue taken between the electrodes (scale bar = 250 µm).

FIGS. 20A-D are micrographs showing the histopathology of rat brain tissue for untreated rats (FIGS. 20A-B) and treated with high-frequency, bipolar pulses at 200 V/250 kHz (FIGS. 20C-D, Rat #2, right hemisphere), with the delineation between treated and untreated tissue shown in FIG. 20C (black, dotted line).

FIGS. 21A-F are MRIs of lesions in rat brain appearing as focal hyper-intense regions (white) compared to adjacent untreated cerebrocortical tissue (gray). FIGS. 21A-C were obtained from Rat #3, in which both the left and right cerebral hemispheres were treated with high-frequency waveforms at 300 V/250 kHz and 400 V/250 kHz, respectively. FIGS. D-F were obtained from Rat #4, which underwent high-frequency, bipolar pulses in the right cerebrum at 400 V/500 kHz, and conventional IRE with unipolar pulses at 50 V in the left cerebrum.

FIGS. 22A-D are data recordings of acceleration (a) versus time during treatments with unipolar IRE pulses and high-frequency IRE pulses.

FIG. 23 is a chart showing peak acceleration (a) during pulsing protocols averaged over the first 90 pulses.

FIGS. 24A-C are schematic diagrams showing electric field, norm (V/cm) contours predicted by the FEM during a 1000 V amplitude burst with a center frequency of 1 kHz (FIG. 24A) and 1 MHz (FIG. 24B). In FIG. 24C, the homogeneous solution is shown for a constant pulse.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Despite being a well-known technique, there is significant controversy about the mechanisms governing electroporation. Weaver, J.C., Electroporation of cells and tissues. IEEE Transactions on Plasma Science, 2000. 28(1): p. 24-33. Even though the biophysical phenomenon at the molecular level is not known, the hypothesis is that in the presence of an externally applied electric field, the lipid bilayer in cellular membranes rearranges to create water-filled structures. These structures (or pores) provide a pathway for ions and molecules through the membranes that normally are impermeable. The dynamics of membrane poration is considered a four-step process: pore induction, expansion, stabilization and resealing. Weaver, J.C. and Y.A. Chizmadzhev, Theory of electroporation: a review. Bioelectrochem. Bioenerg., 1996. 41: p. 135-60. Initial thermal fluctuations are responsible for the presence of hydrophobic pores. There exists a critical radius where it is more energetically favorable for a hydrophobic pore to transition to a hydrophilic pore. In addition, increasing the TMP reduces this critical radius and increases the stability of a hydrophilic pore. Kinosita, K., Jr., S. Kawato, and A. Ikegami, A theory of fluorescence polarization decay in membranes. Biophys J, 1977. 20(3): p. 289-305. When the pore reaches this meta-stable state, it becomes permeable to small molecules. The presence of the induced transmembrane potential lowers the energy required for the pore’s existence. Freeman, S.A., M.A. Wang, and J.C. Weaver, Theory of Electroporation of Planar Bilayer-Membranes - Predictions of the Aqueous Area, Change in Capacitance, and Pore-Pore Separation. Biophysical Journal, 1994. 67(1): p. 42-56. When the electric field has been turned off, the membrane starts to return to its normal membrane potential and resealing of the pores takes place.

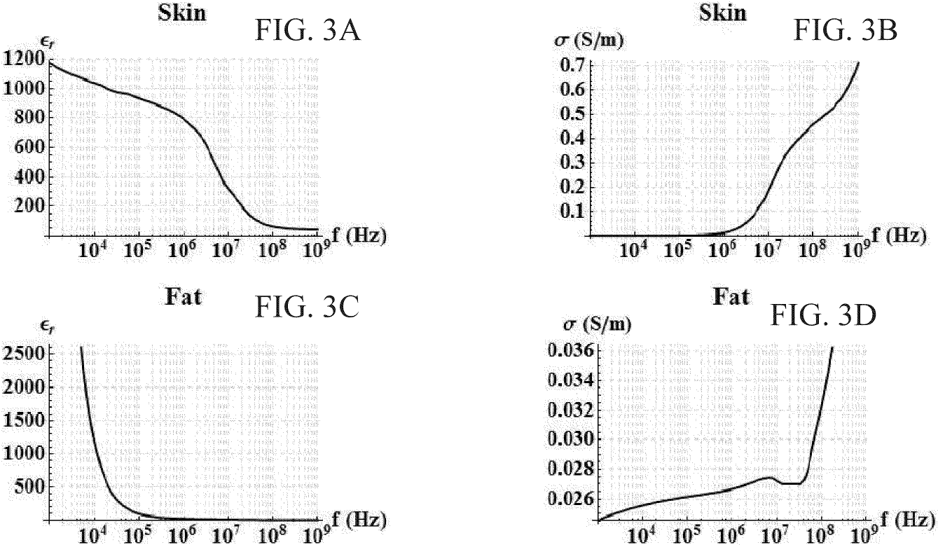

The dielectric permittivity and conductivity of a given tissue are typically functions of frequency. A comparison of the dielectric properties between skin and fat is presented in Table 1. This data was obtained by interpolating the results from Gabriel et al. (FIGS. 3A-D). Gabriel, S., R.W. Lau, and C. Gabriel, The dielectric properties of biological tissues .2. Measurements in the frequency range 10 Hz to 20 GHz. Physics in Medicine and Biology, 1996. 41(11): p. 2251-2269. At varying frequencies, different mechanisms of charge transfer contribute differently to the permittivity and conductivity. Stoy, R.D., K.R. Foster, and H.P. Schwan, Dielectric properties of mammalian tissues from 0.1 to 100 MHz: a summary of recent data. Phys Med Biol, 1982. 27(4): p. 501-13.

TABLE 1

Conductivity of skin and fat as a function of frequency

| Frequency | Waveform | Property | Tissue | |
|---|---|---|---|---|
| | | | Skin | Fat |
| 250 kHz | [image] | σ [S/m] | 0.00216 | 0.0263 |
| | | εᵣ | 888 | 47 |
| 500 KHz | [image] | σ [S/m] | 0.00485 | 0.0265 |
| | | εᵣ | 851 | 33 |
| 1 MHz | [image] | σ [S/m] | 0.0119 | 0.0267 |
| | | εᵣ | 792 | 25 |
| 2 MHz | [image] | σ [S/m] | 0.0290 | 0.0270 |
| | | εᵣ | 700 | 20 |

In general, as the frequency increases, so does the conductivity of the skin and fat. According to Table 1, the difference in conductivity between skin (s) and fat (ƒ) is reduced as the frequency increases from 250 kHz to 2 MHz (σₛ/σ_{f} ~ 1).

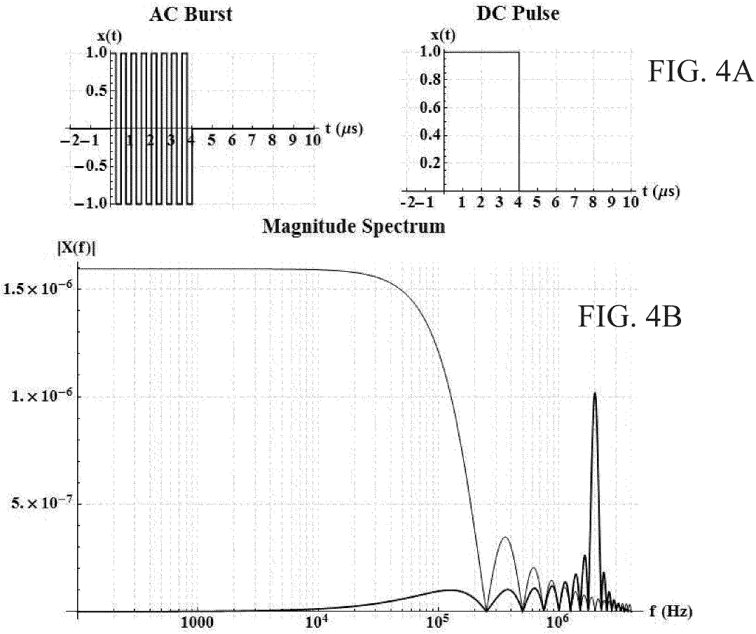

Therefore, if electroporation is used to treat a tumor within a heterogeneous skin fold geometry, the electric field distribution in the surrounding skin and fat would be more homogenous if high-frequency waveforms are utilized. Oftentimes tissue impedance has patient-to-patient variability and the impedance distribution and any impedance changes may be difficult to determine for a particular patient. This point is emphasized further in EXAMPLE 1. Because rectangular waveforms are comprised of components with various frequencies and amplitudes, tissue properties at frequencies associated with the center frequency, defined as the inverse of twice the duration of single polarity, are chosen when studying AC bursts. This is illustrated in FIGS. 4A-B. By taking the absolute value of the Fourier Transform of an AC burst and a DC pulse, the magnitude spectrum can be obtained. While the DC pulse transmits a majority of its power at low frequencies (0 Hz), the AC burst has a characteristic peak at the center frequency (2 MHz in this case).

The benefits of bipolar pulses have been studied for electroporation applications at the single-cell level. Theoretically, Talele et al. have shown that asymmetrical electroporation due to the resting TMP (~0.1 V) (see Gowrishankar 2006) of cells seen when unipolar pulses are delivered (see Chang, D.C., Cell Poration and Cell-Fusion Using an Oscillating Electric-Field. Biophysical Journal, 1989. 56(4): p. 641-652, “Chang 1989”; and Tekle, E., R.D. Astumian, and P.B. Chock, Electroporation by Using Bipolar Oscillating Electric-Field - an Improved Method for DNA Transfection of Nih 3t3 Cells. Proceedings of the National Academy of Sciences of the United States of America, 1991. 88(10): p. 4230-4234, “Tekle 1991”) can be alleviated by switching to bipolar pulses. Talele, S. and P. Gaynor, Non-linear time domain model of electropermeabilization: Response of a single cell to an arbitrary applied electric field. Journal of Electrostatics, 2007. 65(12): p. 775-784. Experimentally, this leads to increased efficiency of macromolecule uptake through the membrane. Chang 1989; and Tekle 1991. Depending on the extracellular conductivity, bipolar pulses with a frequency of 1 MHz (i.e. 500 ns duration of single polarity) can also lessen the dependence of electroporation on cell size, allowing more cells to be electroporated. Talele, S. and P. Gaynor, Non-linear time domain model of electropermeabilization: Effect of extracellular conductivity and applied electric field parameters. Journal of Electrostatics, 2008. 66(5-6): p. 328-334; and Talele, S., P. Gaynor, M.J. Cree, and J. van Ekeran, Modelling single cell electroporation with bipolar pulse parameters and dynamic pore radii. Journal of Electrostatics, 2010. 68(3): p. 261-274. In general, pore formation increases as long as the TMP is sustained above a critical threshold (~1 V). Gowrishankar 2006. Bipolar pulses require higher field strengths to induce a given TMP as compared to a unipolar pulse of equivalent duration. This is accentuated when the frequency of the bipolar pulses is increased, because the time interval above the critical TMP is reduced. Talele, S., P. Gaynor, M.J. Cree, and J. van Ekeran, Modelling single cell electroporation with bipolar pulse parameters and dynamic pore radii. Journal of Electrostatics, 2010. 68(3): p. 261-274. Kotnik et al. have explored the benefits of bipolar pulse trains at significantly lower frequencies, up to 1 kHz (i.e. 500 µs duration of single polarity). At lower frequencies, theoretical results show that the pore formation symmetry can also be normalized with bipolar pulses. Kotnik, T., L.M. Mir, K. Flisar, M. Puc, and D. Miklavcic, Cell membrane electropermeabilization by symmetrical bipolar rectangular pulses. Part I. Increased efficiency of permeabilization. Bioelectrochemistry, 2001. 54(1): p. 83-90, “Kotnik I 2001.” Experimentally, bipolar pulses reduce electrolytic contamination (see Kotnik, T., D. Miklavcic, and L.M. Mir, Cell membrane electropermeabilization by symmetrical bipolar rectangular pulses. Part II. Reduced electrolytic contamination. Bioelectrochemistry, 2001. 54(1): p. 91-5) and the required field strength for reversible electroporation, while the field strength required for IRE remains unchanged. Kotnik I 2001. The authors attribute this to the fact that when the duration of single polarity is much longer than the plasma membrane charging time, permeabilized area differences on the membrane between unipolar and bipolar pulses decreases as pulse amplitude increases.

Bipolar pulse delivery has been studied in vivo for reversible applications of electroporation using center frequencies that are two orders of magnitude lower than that used in embodiments of the present invention. Daskalov et al. have demonstrtated that pulses delivered at 1 kHz are associated with less patient pain in during electrochemotherapy. Daskalov, I., N. Mudrov, and E. Peycheva, Exploring new instrumentation parameters for electrochemotherapy-Attacking tumors with bursts of biphasic pulses instead of single pulses. IEEE Eng Med Biol Mag, 1999. 18(1): p. 62-66. Similarly, Nikolova et al. has recently demonstrated the same findings during electrochemotherapy with a Bacillus Calmette-Guerin vaccine. Nikolova, B., I. Tsoneva, and E. Peycheva, Treatment of Melanoma by Electroporation of Bacillus Calmette-Guerin. Biotechnology & Biotechnological Equipment, 2011. 25(3): p. 2522-2524. Both authors attribute the reduction in patient pain due to the associated reduction in muscle contractions seen with bipolar pulses.

There is a balance between employing pulses that are delivered at a high enough frequency to reduce the conductivity mismatch between different tissues but have a duration of single polarity long enough to induce electroporation of cells comprising the tissues. As mentioned, electrical current associated with pulses longer than ~1 µs is confined to extracellular spaces prior to the onset of electroporation. Ivorra, A., ed. Tissue Electroporation as a Bioelectric Phenomenon: Basic Concepts. Irreversible Electroporation, ed. B. Rubinsky. 2010, Springer Berlin Heidelberg. 23-61; and Esser, A.T., K.C. Smith, T.R. Gowrishankar, and J.C. Weaver, Towards solid tumor treatment by irreversible electroporation: intrinsic redistribution of fields and currents in tissue. Technol Cancer Res Treat, 2007. 6(4): p. 261-74. This can be attributed to the migration of charges towards biological membranes following the application of an external electric field. The time required for a membrane to become charged to 63% of its steady state value is defined as the charging time constant of the membrane. Displacement currents across the plasma membrane allow organelles to be exposed to fields during the time that it takes the plasma membrane to reach steady state. Esser, A.T., K.C. Smith, T.R. Gowrishankar, and J.C. Weaver, Towards Solid Tumor Treatment by Nanosecond Pulsed Electric Fields. Technology in Cancer Research & Treatment, 2009. 8(4): p. 289-306. Once steady state is achieved, the counter-field developed along the plasma membrane due to the accumulation of charges is significant enough to shield the field from entering the cell, and current is directed through extracellular spaces. Only after permeabilization of the membrane does ionic conduction allow the field to re-enter the cell. Kolb, J.F., S. Kono, and K.H. Schoenbach, Nanosecond pulsed electric field generators for the study of subcellular effects. Bioelectromagnetics, 2006. 27(3): p. 172-187. If extracellular current pathways between cells are reduced, as in layers of epithelial cells connected by tight or gap junctions (see Jones, D.M., R.H. Smallwood, D.R. Hose, B.H. Brown, and D.C. Walker, Modelling of epithelial tissue impedance measured using three different designs of probe. Physiological Measurement, 2003. 24(2): p. 605-623), the field is highly concentrated across the layer, and the extent of electroporation in underlying cells is reduced. This problem is alleviated when the duration of single polarity approaches the membrane time constant.

By treating cells as a series of spherical, dielectric shells containing and surrounded by a conductive medium, the analytical solution for induced TMP across the plasma membrane (ΔΦ) can be obtained according to the law of total current (see Yao, C.G., D.B. Mo, C.X. Li, C.X. Sun, and Y. Mi, Study of transmembrane potentials of inner and outer membranes induced by pulsed-electric-field model and simulation. IEEE Trans Plasma Sci, 2007. 35(5): p. 1541-1549, “Yao 2007”):

$$∇ ⋅ \left({ε}_{0} {ε}_{r} \frac{\partial E}{\partial t} + σ E\right) = {\text{Λ}}_{k} ∇ ⋅ E = 0$$

$${\text{Λ}}_{k} = σ + {ε}_{0} {ε}_{r} \frac{\partial }{\partial t}$$

where A is the admittivity operator and the subscript k denotes cellular regions including the nucleoplasm (n), nuclear envelop (ne), cytoplasm (c), plasma membrane (pm), and extracellular space (e). Transforming (2), (5), and (6) into the frequency domain (see Yao 2007):

$$E = - ∇ \text{Φ} \left(s\right)$$

$${\text{Λ}}_{k} ∇ ⋅ E \left(s\right) = 0$$

$${\text{Λ}}_{k} \left(s\right) = σ + {ε}_{0} {ε}_{r} s$$

where s = jω = j2πƒ, and applying the appropriate boundary conditions of potential continuity and normal vector continuity of current density at the interface between the different regions yields the solution for TMP (see Yao 2007):

$$\text{ΔΦ} \left(s\right) = F \left({\text{Λ}}_{n} , {\text{Λ}}_{n e} , {\text{Λ}}_{c} , {\text{Λ}}_{p m} , {\text{Λ}}_{e}\right) E \left(s\right) cos θ$$

where θ represents the polar angle at the cell center between the electric field and the point of interest along the membrane. TMP is defined as the potential directly outside the membrane minus the inside. The natural, resting component of the plasma membrane TMP was ignored in all simulations, because it is typically an order of magnitude less than the induced TMP. See Gowrishankar 2006. Further, the TMP across the nuclear envelope never reached a permeabilizing threshold with the chosen pulsing protocols, and reference to TMP from this point forward refers only to the plasma membrane. As shown in Table 2, the term F(Λₖ) represents a transfer function of the TMP that reflects the geometric and dielectric properties of the cellular regions as a function of the admittivity. See Hu, Q., S. Viswanadham, R.P. Joshi, K.H. Schoenbach, S.J. Beebe, and P.F. Blackmore, Simulations of transient membrane behavior in cells subjected to a high-intensity ultrashort electric pulse. Physical Review E, 2005. 71(3). Dielectric properties at the cellular level are assumed to be frequency independent, which is valid for predicting TMP up to around 100 MHz. Kotnik, T. and D. Miklavcic, Theoretical evaluation of the distributed power dissipation in biological cells exposed to electric fields. Bioelectromagnetics, 2000. 21(5): p. 385-394.

TABLE 2

Dielectric properties of various cellular regions.

| Geometry | σ [S/m] | ε_{γ} | Dimensions [m] |
|---|---|---|---|
| Extracellular Space | 0.6 | 80.0 | - |
| Plasma Membrane | 5.3 × 10⁻⁶ | 7.0 | 7.0 × 10⁻⁹ (thickness) |
| Cytoplasm | 0.13 | 60.0 | 5.0 × 10⁻⁶ (radius) |
| Nuclear Envelope | 4.3 × 10⁻³ | 22.8 | 40.0 × 10⁻⁹ (thickness) |
| Nucleoplasm | 0.18 | 120.0 | 2.5 × 10⁻⁶ (radius) |

The exact formulation for F(Λₖ) is lengthy and can be found in (see Kotnik, T. and D. Miklavcic, Theoretical evaluation of voltage inducement on internal membranes of biological cells exposed to electric fields. Biophysical Journal, 2006. 90(2): p. 480-491), but is not included here for brevity. The term E(s) represents the Laplace transform of the pulsed electric field as a function of time.

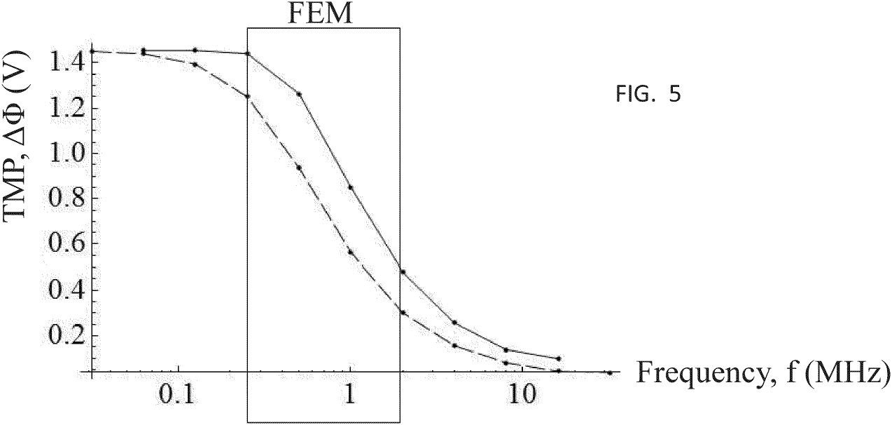

Using the analytical model, the frequency dependence of the induced TMP can be investigated for both rectangular and sinusoidal electric fields with identical maximum amplitude. By substituting the transient electric fields into (6) the results of a parametric study on TMP for frequencies spanning from 62.5 kHz to 16 MHz are shown in FIG. 5. The maximum amplitude of the sinusoidal and bipolar rectangular electric fields was 2000 V/cm (peak). For this applied field and the given geometric and dielectric properties of the modeled cell, the TMP never exceeds 1.46 V. Additionally, the time constant of the plasma membrane is 345 ns. All measurement were taken at the cell pole (θ = 0) to depict the maximum achieved TMP after the system reached a steady oscillatory state. From the curve, as the frequency increases, the magnitude of the TMP is reduced. For the sinusoidal waveform, the reduction is evident at lower frequencies compared to the rectangular waveform. This has to do with the fact that the rectangular waveform maintains its maximum amplitude for a longer period of time than the sinusoidal waveform. It is not until the frequency of the rectangular waveform surpasses 250 kHz that a dramatic decrease in TMP occurs. For this reason, only rectangular pulses in a frequency window of 250 kHz to 2 MHz are best suited for electroporation with high-frequency, bipolar pulses.

Based on the analytical model for TMP presented above, the time constant of the plasma membrane for a constant field (2000 V/cm) is 345 ns. The time constant of 345 ns falls between the 2 MHz (250 ns pulse duration) and 1 MHz (500 ns pulse duration) bursts. Further, the 500 kHz burst (1 µs pulse duration) is close to the time it takes the TMP to reach steady state. As frequency is increased, the dielectric properties different tissues become more macroscopically homogeneous, but above 2 MHz, the pulse duration is not adequate for the cell to charge and induce electroporation. According to in vitro experiments that utilize bipolar rectangular pulses, the typical burst width required to induce either reversible electroporation or IRE increases with the frequency of the applied field. For EGT, a 60 kHz bipolar square with a burst width of 400 µs and an amplitude of 1600 V/cm has a six times greater transfection efficiency than a 1 MHz bipolar square wave with equal amplitude and width. Tekle, E., R.D. Astumian, and P.B. Chock, Electroporation by Using Bipolar Oscillating Electric-Field - an Improved Method for DNA Transfection of Nih 3t3 Cells. Proceedings of the National Academy of Sciences of the United States of America, 1991. 88(10): p. 4230-4234 (Telke 1991). In terms of IRE, a 60 kHz bipolar square with a burst width of 400 µs and an amplitude of 4000 V/cm results in 19 % cell viability. Telke 1991. These results were obtained when a single burst was delivered to the sample. The inventors, however, appear to be the first in providing data on high-frequency electroporation with rectangular pulses that implemented multiple bursts. Similar to how multiple unipolar pulses are typically delivered in ECT, EGT, or IRE protocols to enhance the desired outcome (see Belehradek, J., S. Orlowski, L.H. Ramirez, G. Pron, B. Poddevin, and L.M. Mir, Electropermeabilization of Cells in Tissues Assessed by the Qualitative and Quantitative Electroloading of Bleomycin. Biochimica Et Biophysica Acta-Biomembranes, 1994. 1190(1): p. 155-163; and Garcia, P.A., J.H. Rossmeisl, R.E. Neal, T.L. Ellis, J.D. Olson, N. Henao-Guerrero, J. Robertson, and R.V. Davalos, Intracranial Nonthermal Irreversible Electroporation: In Vivo Analysis. Journal of Membrane Biology, 2010. 236(1): p. 127-136) multiple bipolar bursts would likely produce similar trends. Data is also available for burst sinusoidal waveforms in the frequency range of 2 kHz to 50 MHz (see Jordan, D.W., R.M. Gilgenbach, M.D. Uhler, L.H. Gates, and Y.Y. Lau, Effect of pulsed, high-power radiofrequency radiation on electroporation of mammalian cells. Ieee Transactions on Plasma Science, 2004. 32(4): p. 1573-1578; and Katsuki, S., N. Nomura, H. Koga, H. Akiyama, I. Uchida, and S.I. Abe, Biological effects of narrow band pulsed electric fields. Ieee Transactions on Dielectrics and Electrical Insulation, 2007. 14(3): p. 663-668), but the results are inconclusive, and sinusoidal waveforms are less efficient than rectangular bipolar pulses for inducing electroporation. Kotnik, T., G. Pucihar, M. Rebersek, D. Miklavcic, and L.M. Mir, Role of pulse shape in cell membrane electropermeabilization. Biochimica Et Biophysica Acta-Biomembranes, 2003. 1614(2): p. 193-200.

There is a narrow window of pulse parameters where ECT and EGT have proven to be effective without reducing cell viability by IRE. For ECT, the field for inducing optimal reversible electroporation conditions is between 300 and 500 V/cm in tumors, when eight square-wave pulses 100 µs in duration are delivered at a frequency of 1 Hz. Mir, L.M., Therapeutic perspectives of in vivo cell electropermeabilization. Bioelectrochemistry, 2001. 53: p. 1-10 (Mir 2001). For EGT, permeabilization conditions are optimal when eight square-wave pulses 20 ms in duration are delivered at a frequency of 1 Hz, which constitutes a field of around 90 V/cm. Mir 2001. To maintain its non-thermal benefits, the pulse parameters for IRE procedures are restricted to those that minimize any associated Joule heating. Davalos, R.V. and B. Rubinsky, Temperature considerations during irreversible electroporation. International Journal of Heat and Mass Transfer, 2008. 51(23-24): p. 5617-5622. However, a similar field strength and duration to those required for ECT can induce IRE when the number of pulses is raised above the traditional 8 pulses to 90 pulses, and the temperature of the tissue remains below 50° C. Rubinsky, J., G. Onik, P. Mikus, and B. Rubinsky, Optimal Parameters for the Destruction of Prostate Cancer Using Irreversible Electroporation. Journal of Urology, 2008. 180(6): p. 2668-2674.

In addition to being bipolar, the pulses used according to methods of the invention can have a duration of single polarity (~1 µs) that is two orders of magnitude less than the duration of a conventional electroporation pulse (~100 µs) and an amplitude that is one order of magnitude less than supraporation protocols with nanosecond pulsed electric field (nsPEF). Supraporation involves pulses with a duration ranging from 1-100 ns and an amplitude ranging from 10-100 kV/cm. These electric fields are capable of causing electroporation within the membranes of intracellular organelles. Vernier, P.T., Y.H. Sun, and M.A. Gundersen, Nanoelectropulse-driven membrane perturbation and small molecule permeabilization. Bmc Cell Biology, 2006. 7. When the pulse length is shorter than the charging time of the plasma membrane, the field can penetrate the plasma membrane to reach the cell interior. Beebe, S.J., P.M. Fox, L.J. Rec, L.K. Willis, and K.H. Schoenbach, Nanosecond, high-intensity pulsed electric fields induce apoptosis in human cells. FASEB J, 2003. 17(9): p. 1493-5. Because organelles are smaller in diameter than cells, the amplitude required to raise the TMP on organelles up to ~1 V is greater than that in ECT and IRE procedures. However, due to the ultra-short nature of the pulses, the accompanying Joule heating is still negligible. Schoenbach, K.H., S.J. Beebe, and E.S. Buescher, Intracellular effect of ultrashort electrical pulses. Bioelectromagnetics, 2001. 22(6): p. 440-8. While immediate necrosis is suspected as the primary mechanism of cell death following IRE, apoptosis triggered by DNA fragmentation and the release of calcium from intracellular stores occurs in cells exposed to sufficiently high nsPEFs. Beebe, S.J., J. White, P.F. Blackmore, Y.P. Deng, K. Somers, and K.H. Schoenbach, Diverse effects of nanosecond pulsed electric fields on cells and tissues. DNA and Cell Biology, 2003. 22(12): p. 785-796.

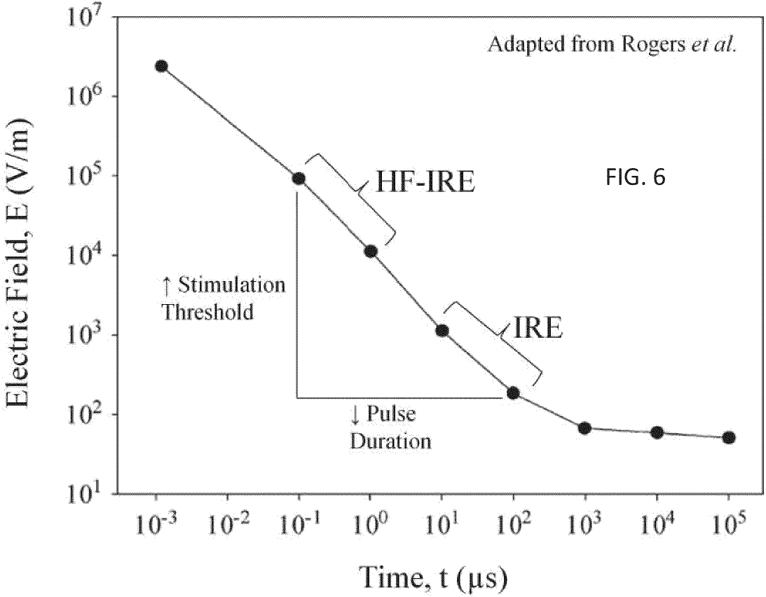

In vivo experiments on supraporation have shown that the ultra-short, unipolar pulses do not cause stimulation of excitable tissue, such as muscle and nerves. Long, G., P.K. Shires, D. Plescia, S.J. Beebe, J.F. Kolb, and K.H. Schoenbach, Targeted Tissue Ablation With Nanosecond Pulses. Ieee Transactions on Biomedical Engineering, 2011. 58(8). This is a consequence of the pulses being below the strength-duration threshold determined by Rogers et al. Rogers, W.R., J.H. Merritt, J.A. Comeaux, C.T. Kuhnel, D.F. Moreland, D.G. Teltschik, J.H. Lucas, and M.R. Murphy, Strength-duration curve for an electrically excitable tissue extended down to near 1 nanosecond. Ieee Transactions on Plasma Science, 2004. 32(4): p. 1587-1599. As seen in FIG. 6, for IRE pulses, the electric field threshold for stimulation is between 1-10 V/cm. The present invention describes pulses where the duration of single polarity is as low as 100 ns. At this duration, the electric field threshold for stimulation increases to 1000 V/cm, which is above the amplitude required for reversible electroporation and on the order of the amplitude for IRE.

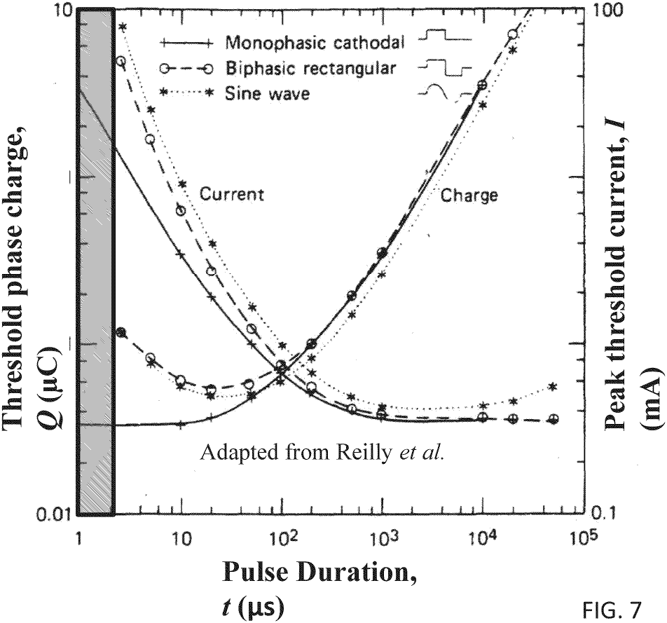

In addition to the duration of single polarity being reduced, the fact that the inventive waveforms are inherently bipolar offers an additional benefit in terms of the stimulation of excitable tissue. As shown in FIG. 7, biphasic waveforms have a higher threshold current for inducing nerve stimulation. Reilly, J.P., V.T. Freeman, and W.D. Larkin, Sensory Effects of Transient Electrical-Stimulation - Evaluation with a Neuroelectric Model. IEEE Trans Biomed Eng, 1985. 32(12): p. 1001-1011. Further, the threshold increases exponentially as the duration of single polarity is decreased. While the mechanism of this phenomenon is unknown, it is thought that the reversal in polarity prevents an action potential from being generated by limiting the flow of sodium ions down their concentration gradient. This has been shown to translate to a reduced muscle twitch force during bipolar functional electrical stimulation as opposed to monopolar. Vandenhonert, C. and J.T. Mortimer, Response of the Myelinated Nerve-Fiber to Short Duration Biphasic Stimulating Currents. Annals of Biomedical Engineering, 1979. 7(2): p. 117-125.

The inventors have shown that bipolar waveforms can induce IRE at center frequencies high enough to eliminate muscle contraction completely. This procedure is termed high-frequency IRE (H-FIRE). Overall, the results indicate that H-FIRE can produce more predictable treatment outcomes, reduce the potential for thermal damage, and obviate the need for (or reduce the necessity of) neuroparalytic agents delivered prior to or during treatment.

The following examples show that bursts of bipolar, nanosecond pulses can maintain a critical TMP beneath epithelial layers, while minimizing Joule heating. This has to do with the ability of high-frequency waveforms to achieve a macroscopically homogeneous field distribution in a heterogeneous system. At high-frequencies, tissues with a low passive DC conductivity become more conductive. Additionally, it is proven that high-frequency IRE (H-FIRE) can be applied to non-thermally ablate tissue while eliminating muscle contractions seen in conventional IRE protocols with longer duration unipolar pulses. These results have implications not only for skin, brain, and liver as presented here, but for other tissues, such as bone, breast, pancreas, kidney, and lung. These examples should not be considered as limiting the invention in any way.

As a general background to the examples, it is noted that the inventors and their colleagues have successfully demonstrated that finite element models (FEMs) can accurately predict treatment outcomes of pulsed electric field therapies for cancer treatment. See Edd, J.F. and R.V. Davalos, Mathematical modeling of irreversible electroporation for treatment planning. Technol Cancer Res Treat, 2007. 6: p. 275-286; and Edd, J.F., L. Horowitz, R.V. Davalos, L.M. Mir, and B. Rubinsky, In vivo results of a new focal tissue ablation technique: irreversible electroporation. IEEE Trans Biomed Eng, 2006. 53(7): p. 1409-15.

### Example 1: High-Frequency Electroporation Results in More Uniform and Predictable Treatment Outcomes in Heterogeneous Tissues

A 2D axisymmetric FEM representative of a cylindrical section of non-infiltrated fat encapsulated by dry skin was simulated using COMSOL 3.5a (Burlington, MA). The electric potential distribution within the tissue was obtained by transiently solving:

$$- ∇ ⋅ \left(σ ∇ \text{Φ}\right) - {ε}_{0} {ε}_{r} ∇ ⋅ \left(\frac{\partial ∇ \text{Φ}}{\partial t}\right) = 0$$

where Φ is the electric potential and σ and εᵣ are the conductivity and relative permittivity of each tissue layer, respectively, which depends on frequency (Table 1). Equation (7) is obtained from Maxwell’s equations assuming no external current density (J = σE), no remnant displacement (D = ε₀εᵣE), and the quasi-static approximation. This approximation implies a negligible coupling between the electric and magnetic fields (∇×E=0), which allows for the expression of electric field only in terms of electric potential:

$$E = - ∇ ϕ$$

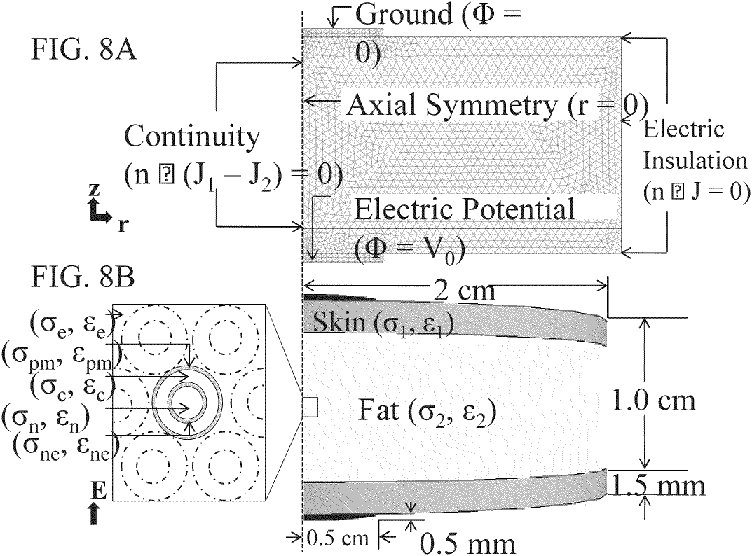

Dielectric properties of the bulk tissue were chosen from data generated by Gabriel et al. (see Gabriel, S., R.W. Lau, and C. Gabriel, The dielectric properties of biological tissues .2. Measurements in the frequency range 10 Hz to 20 GHz. Physics in Medicine and Biology, 1996. 41(11): p. 2251-2269) available at (http://niremf.ifac.cnr.it/docs/dielectric/home.html). The data was interpolated in Mathematica 7 (Wolfram Research, Inc.) in order to estimate the dielectric properties at the desired frequencies. Dielectric properties of the electrode were chosen to be stainless steel, as incorporated in the Comsol material library. All electrical boundary conditions are shown in FIGS. 8A-B, which provides in FIG. 8A, a meshed geometry of the FEM with boundary settings. The mesh consists of 3028 elements and was refined until there was < 0.1% change in the magnitude of the electric field at the center of the tissue. FIG. 8B provides a schematic diagram of the geometry with dimensions. The box represents an expanded view of the tissue that describes the link between the macroscopic electric field (E) and the microscopic analysis of TMP. Adjacent cells are drawn with dashed lines, indicating their role was ignored in calculating TMP.

Because rectangular waveforms are comprised of components with various frequencies and amplitudes, tissue properties at frequencies associated with the center frequency, defined as the inverse of twice the duration of single polarity, are chosen. Intuitively, the duration of single polarity defines the frequency at which the current changes direction in the tissue. The pulses were constructed by multiplying the applied voltage by a function consisting of two smoothed Heaviside functions with a continuous second derivative and a tolerance of 5 ns (rise and fall times). The quasi-static assumption is confirmed based on the fact that the primary frequency of the pulses is lower than 200 MHz (rise and fall times), which corresponds to a wavelength that is greater than the longest dimension in the geometry. Chen, M.T., C. Jiang, P.T. Vernier, Y.H. Wu, and M.A. Gundersen, Two-dimensional nanosecond electric field mapping based on cell electropermeabilization. PMC Biophys, 2009. 2(1): p. 9. The inclusion of a permittivity term in (1) differs from previous, simplified models (see Edd, J.F. and R.V. Davalos, Mathematical Modeling of irreversible Electroporation for treatment planning. Technology in Cancer Research & Treatment, 2007. 6(4): p. 275-286; and Neal, R.E. and R.V. Davalos, The Feasibility of Irreversible Electroporation for the Treatment of Breast Cancer and Other Heterogeneous Systems. Annals of Biomedical Engineering, 2009. 37(12): p. 2615-2625), and accounts for reactive component of tissue to time dependent pulsing, which is required for obtaining accurate potential distributions in heterogeneous models. Yousif, N., R. Bayford, and X. Liu, The Influence of Reactivity of the Electrode-Brain Interface on the Crossing Electric Current in Therapeutic Deep Brain Stimulation. Neuroscience, 2008. 156(3): p. 597-606.

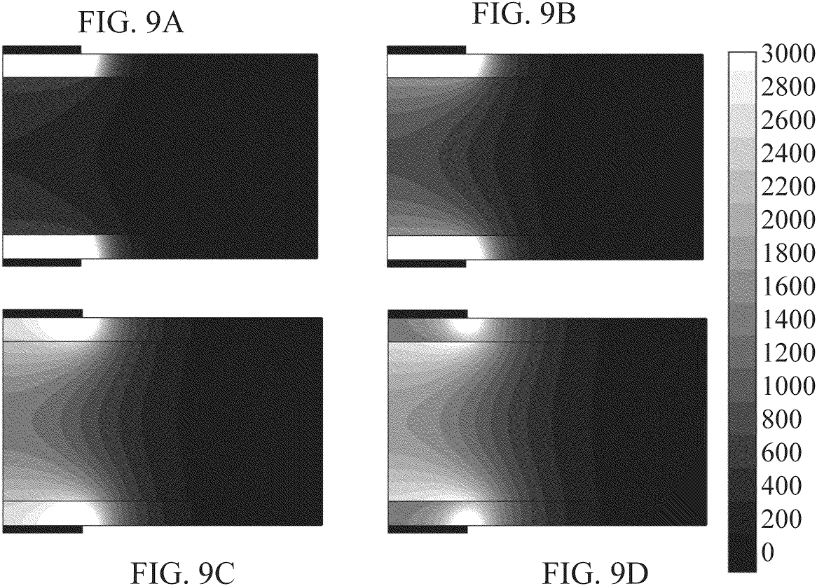

FIGS. 9A-D show the electric field distribution at the end of a 2 µs burst with various frequencies given in Table 1. In each case, the maximum applied voltage was set to 2600 V (peak) in order to set up a voltage to distance ratio of 2000 V/cm between the electrodes (1.3 cm spacing). From the surface contour map, as frequency is increased, the electric field in the fat rises while the field in the skin drops. This trend extends to the point that at 2 MHz the field in the skin is lower than the fat, which is a direct result of the tissue dielectric properties at that frequency (greater conductivity and permittivity of skin as compared to fat). Therefore, high-frequency fields, or pulses with shorter duration, are better suited to penetrate epithelial layers, such as the skin, and reach underlying tissue.

### Example 2: High-Frequency Electroporation Results in Homogeneous Energy Deposition and Reduces the Potential for Thermal Damage in Low Passive Conductivity Tissue

The temperature distribution in the model described in EXAMPLE 1 was obtained by transiently solving a modified version of the Pennes bioheat equation (see Pennes, H.H., Analysis of tissue and arterial blood temperatures in the resting human forearm. J Appl Physiol, 1948. 1(2): p. 93-122) with the inclusion of a Joule heating term:

$$\begin{matrix}ρ C \frac{\partial T}{\partial t} = ∇ ⋅ \left(k ∇ T\right) + {ρ}_{b} {ω}_{b} {C}_{b} \left({T}_{b} - T\right) \\ + {Q}_{m} + \left|J ⋅ E\right|\end{matrix}$$

where T is the tissue temperature, T_{b} is the blood temperature, k is the thermal conductivity of the tissue, C and C_{b} are the tissue and blood specific heat, respectively, ρ and ρ_{b} are the tissue and blood density, respectively, Qₘ is the metabolic heat source term, ω_{b} is the blood perfusion coefficient, and |J·E| is the Joule heating term. All thermal tissue properties are given in Table 3. Fiala, D., K.J. Lomas, and M. Stohrer, A computer model of human thermoregulation for a wide range of environmental conditions: the passive system. Journal of Applied Physiology, 1999. 87(5): p. 1957-1972.

TABLE 3

Thermal tissue properties of various tissues

| Property | Tissue | | |
|---|---|---|---|
| | Blood | Skin | Fat |
| ρ [kg/m³] | 1069 | 1085 | 850 |
| C [J/(Kg□K)] | 3650 | 3680 | 2300 |
| k[W/(m□K)] | - | 0.47 | 0.16 |
| ω[⅟s] | - | 1.1 | 0.0036 |
| Qₘ [kg/m³] | - | 368 | 58 |

Due to the presence of different tissue layers and the high frequencies under consideration (250 kHz - 2 MHz), displacement currents are considered along with conduction currents in the formulation of Joule heating:

$$J = {J}_{D} + {J}_{C} = {ε}_{0} {ε}_{r} \frac{\partial E}{\partial t} + σ E$$

where J is the total current density, J_{D} is the displacement current density, and J_{C} is the conduction current density. In order to ensure that negative current components due to polarity changes add to the total current in the tissue, the absolute value of the resistive heating term was taken prior to temperature calculations. It was assumed that all subdomains were initially at physiologic temperature (To = 310.15 K). The boundaries between the electrode-skin interface and the skin-fat interface were treated as continuous (n·(k₁∇T₁ - k₂∇T₂) = 0), the centerline was defined as axial symmetry (r = 0), and the remaining boundaries were thermally insulated (n·(k∇T) = 0) for conservative temperature estimates. Temperature profiles were investigated along the centerline (r = 0 mm) in the middle of the fat (z = 0 mm) and skin (z = 5.75 mm) layers. Data was imported into Mathematica, and a moving average with a period of 100 ns was taken to smooth the plots. Additionally, the data was fit with a linear trendline in order to extrapolate to longer burst widths and predict the onset of thermal damage.

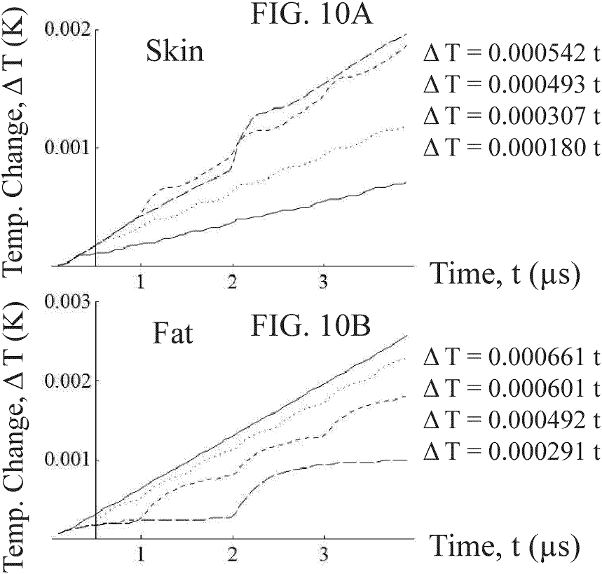

Temperature changes predicted by the FEM at the center of the skin and fat are shown in FIGS. 10A-B, which provides temperature changes predicted by the FEM at the center of the skin (FIG. 10A) and fat (FIG. 10B) for frequencies of 250 kHz (- -), 500 kHz (- - -), 1 MHz (· · ·), and 2 MHz (-). Equations represent a linear fit to the data. In this case, a burst width of 4 µs was simulated in order to capture the trends in temperature development. Polarity of the 2 µs pulse (250 kHz) was switched between pulses to maintain consistency with the other waveforms that are inherently bipolar. With respect to the skin, as the frequency of the applied field increases, the temperature rises at a slower rate. This is a consequence of the fact that the electric field within the skin also decreases with increasing frequency. In the case of the fat, the temperature rises at a faster rate when the frequency of the applied field is increased. At first glance, this seems to be detrimental, however, it is merely an indication that energy is preferentially being deposited more uniformly into the fat at higher frequencies. Again, this can be correlated to the electric field profile. In both tissues, the sharp rises in temperature are due to the spikes in displacement current that occur at the onset and offset of each pulse (data not shown). The total temperature increase in all cases is less than 0.003 K for a burst width of 4 µs. As explained in the discussion, even for bursts of longer widths, the temperature increase is not enough to promote thermal damage.

The onset of protein denaturation and loss of cell structure occurs above 318.15 K (see Bilchik, A.J., T.F. Wood, and D.P. Allegra, Radiofrequency ablation of unresectable hepatic malignancies: Lessons learned. Oncologist, 2001. 6(1): p. 24-33), which correlates to an increase in temperature of 8 K above physiological temperature. Using this information, the maximum energy delivery period (number of pulses multiplied by pulse duration) can be calculated for an amplitude of 2000 V/cm at each of the frequencies investigated using the trendlines generated by the FEM data (FIGS. 9A-D). In the skin layer, heating is reduced by increasing the frequency of the applied field. This shows that the potential for thermal damage in the skin is reduced when the frequency of the applied field is increased. At higher frequencies, the energy is preferentially deposited in the fat layer. For 2 MHz, the total energy delivery period required to cause an 8 K increase in temperature is 12 ms. An example treatment plan can include 12, 1 ms pulses separated by a delay of 1 s. If the frequency is reduced to 500 kHz, which shows the greatest electroporation efficiency (Table 4, see EXAMPLE 3), the allowable energy delivery period increases to 16 ms, which would permit the delivery of an additional 4, 1 ms pulses before the onset of thermal damage.

TABLE 4

Various exemplary treatment protocols.

| Frequency (pulse duration) | Time (µs), \|TMP\| > 0.5 V | % of Pulse, \|TMP\| > 0.5 V |
|---|---|---|
| 250 kHz (2 µs) | 1.2 | 60 |
| 500 kHz (1 µs (×2)) | 1.9 | 95 |
| 1 MHz (500 ns (×4)) | 1.3 | 65 |
| 2 MHz (250 ns (×8)) | 0.1 | 5 |

The restrictions could be increased if less conservative estimates are obtained that account for heat dissipation between pulses and heat convection at the tissue surface. Lackovic, I., R. Magjarevic, and D. Miklavcic, Three-dimensional Finite-element Analysis of Joule Heating in Electrochemotherapy and in vivo Gene Electrotransfer. Ieee Transactions on Dielectrics and Electrical Insulation, 2009. 16(5): p. 1338-1347. These projected protocols represent a maximum, and it is likely that the desired effects will be induced at a significantly lower energy. See Belehradek, J., S. Orlowski, L.H. Ramirez, G. Pron, B. Poddevin, and L.M. Mir, Electropermeabilization of Cells in Tissues Assessed by the Qualitative and Quantitative Electroloading of Bleomycin. Biochimica Et Biophysica Acta-Biomembranes, 1994. 1190(1): p. 155-163; and Garcia, P.A., J.H. Rossmeisl, R.E. Neal, T.L. Ellis, J.D. Olson, N. Henao-Guerrero, J. Robertson, and R.V. Davalos, Intracranial Nonthermal Irreversible Electroporation: In Vivo Analysis. Journal of Membrane Biology, 2010. 236(1): p. 127-136.

### Example 3: High-Frequency Electroporation can Overcome Shielding Effects of Low Passive Conductivity Tissues and Induce Electroporation in Underlying Layers

The analytical model for TMP described in this specification was utilized to investigate electroporation in a hypothetical cell located along the centerline (r = 0 mm) in the middle of the fat (z = 0) and skin (z = 5.75 mm) layers of the FEM described in EXAMPLE 1. The equations for TMP are derived under the assumption that there is no influence on the microscopic electric field from neighboring cells. Therefore, the macroscopic electric field in the bulk tissue predicted by the FEM dictates the microscopic electric field experienced by the cell. The vertical z-component of the electric field was imported from the specific locations within FEM into Mathematica to account for polarity changes. The radial r-component was neglected due to the fact that it never surpassed 3 V/cm as current traveled primarily in the z-direction. Non-uniform electric field data was fit with a series of step functions (50 ns duration), such that the Laplace transform of the field could be performed and the solution for TMP could be obtained in the frequency domain as the summation of individual steps. The inverse Laplace transform of the data was taken to obtain the complete time courses. Measurements were taken at the pole (θ = 0) to depict the maximum induced TMP around the cell.

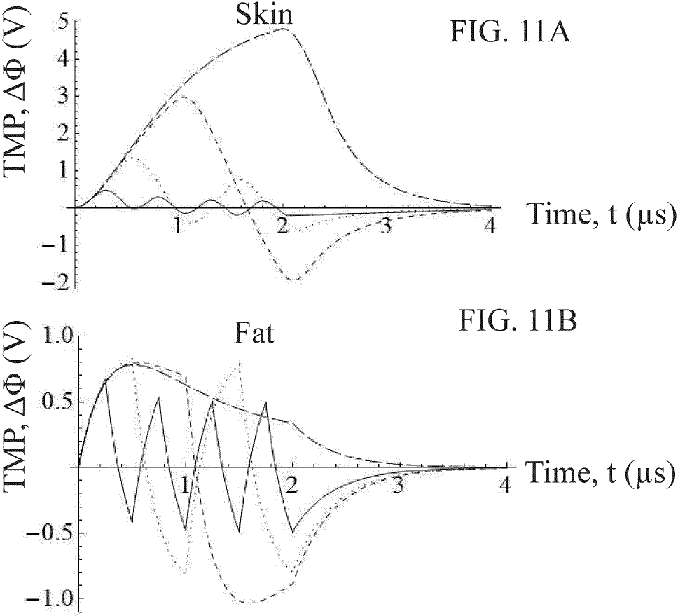

With respect to the skin, as the frequency of the applied field increases, the maximum oscillation amplitude of the TMP decreases, as shown in FIGS. 11A-B. This occurs for two reasons. First, as seen in FIGS. 8A-B, the electric field in the skin decreases with increasing frequency. Second, as seen in FIG. 5, even with constant field amplitude, the TMP decreases with increasing frequency, because the time during which the membrane has to charge before the polarity switches is less at higher frequencies. In the case of the fat, the behavior is slightly more complex. At lower frequencies, a majority of the voltage drop occurs across the skin as demonstrated in FIGS. 9A-D, resulting in a reduced electric field in the fat. This shielding effect is best shown in FIGS. 10A-B along the 250 kHz trace. According to FIG. 5, at 250 kHz, the maximum TMP should be reached. However, due to the shielding effect from the skin, a reduction in the TMP prior to the polarity change is seen. This reduction in TMP can be alleviated by increasing the frequency of the applied field. However, the tradeoff between increased frequency and reduced TMP is still evident at a frequency of 2 MHz (FIGS. 11A-B).

As mentioned, there is a balance between employing pulses that are delivered on a short enough timescale to flow through epithelial cells but are long enough to induce electroporation in underlying cells. The time constant of 345 ns, predicted by the analytical model for TMP, falls between the 2 MHz (250 ns pulse duration) and 1 MHz (500 ns pulse duration) bursts. Further, the 500 kHz burst (1 µs pulse duration) is close to the time it takes the TMP to reach steady state. Table 4 summarizes the results based on the time that the TMP on a hypothetical cell at the center of the fat layer is above 0.5 V. This amplitude was chosen such that even the highest frequency burst was above the set voltage level for a certain amount of time. The results would hold if the applied field was doubled and the voltage level was set to the 1 V threshold for pore formation, due to the linear dependence of TMP on the electric field. Based on this criterion, a frequency of 500 kHz is best suited to treat cells in the fat layer, followed by 1 MHz and 250 kHz. As frequency is increased, the dielectric properties and electric field distribution in the skin and fat become more macroscopically homogeneous, but above 1 MHz, the pulse duration is not adequate for the cell to charge.

### Example 4: System for Implementing High-Frequency, Bipolar Pulses for Tissue Electroporation

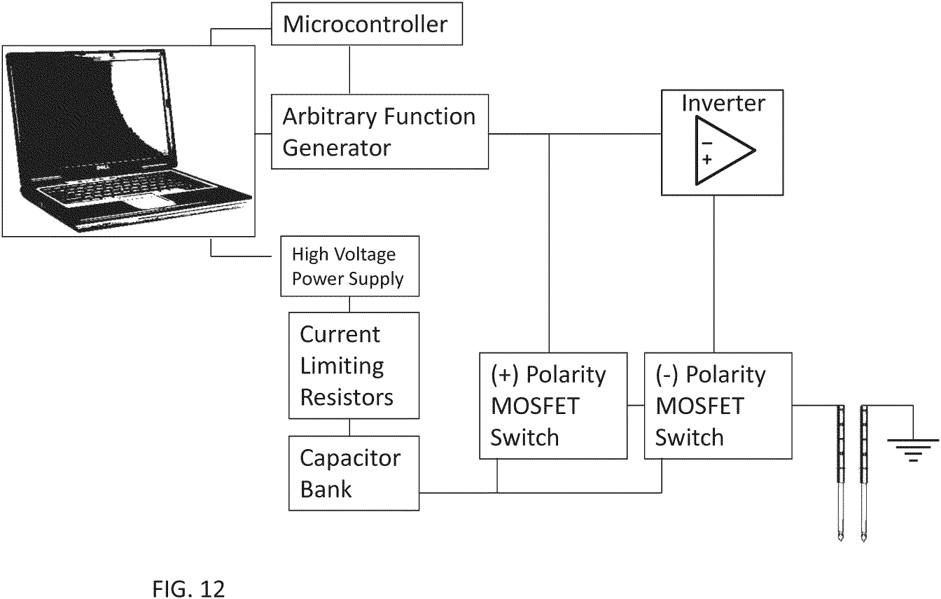

The electronic drive system for delivering bipolar electroporation signals is schematically depicted in FIG. 12. The system relies upon both commercially available components and circuits built by the inventors. An arbitrary function generator (Tektronix AFG 3011) is programmed to output a tri-state square waveform. The AFG 3011 is capable of generating 20 V peak-to-peak into a 50 ohm load and has an effective analog bandwidth of 8 MHz. The burst width, interval between bursts, and total number of bursts is externally controlled by a microcontroller (Arduino Duemilanove) through the general purpose input/output (GPIO) pins. The output signal for a 1 MHz waveform with a burst width of 10 µs and amplitude of 6 V peak is given in FIG. 12. This signal is simultaneously fed through both positive polarity and negative polarity high voltage MOSFET switches (IXYS Colorado HV 1000). The signal into the negative polarity HV 1000 is inverted using an LM 7171 op amp with a slew rate of 4100 V/ µs in order to properly sequence the amplification of the positive and negative polarity pulses without delay. The maximum output of each HV 1000 is 17 A and +/- 850 V into a 50 ohm load. Additionally the pulse rise time is 10 ns or less. This results in an amplification of the AFG 3011 trigger signal up to 1700 V peak-to-peak, which is capable of inducing electroporation when the electrodes are spaced approximately 3 cm apart or less. The input power to each HV 1000 is maintained by a high voltage sequencer (LabSmith HVS 448), which can regulate voltage up to +/- 3000 V and current up to 100 mA. In order to increase current storage up to 17 A, an external capacitor bank was included between the HVS 448 and HV 1000. The total capacitance of the bank can be adjusted depending on the desired voltage and current output or electrode spacing. This system allows for a flexible treatment program that may be tailored to meet a patient’s individual needs.

Other systems are available in the literature for generating bipolar pulses, and the invention should not be limited to the system described above. For example, De Vuyst et al. built a generator around an NE555 timer configured as an astable multivibrator capable of producing up to 50 kHz bipolar pulses. De Vuyst, E., M. De Bock, E. Decrock, M. Van Moorhem, C. Naus, C. Mabilde, and L. Leybaert, In situ bipolar Electroporation for localized cell loading with reporter dyes and investigating gapjunctional coupling. Biophysical Journal, 2008. 94(2): p. 469-479. However, the frequency of the pulses administered according to embodiments of the invention are an order of magnitude greater, which is easily met by the bandwidth of the AFG3011. Additionally, the MOSFET switches provide an excellent means to produce high-frequency pulses for high voltage switching. However, MOSFETs are not the only semiconductor devices that can be utilized to produce a pulse. Bipolar Junction Transistors (BJTs), Insulated Gate Bipolar Transistors (IGBTs), and Junction Field Effect Transistors (JFETs) are examples of some of the semiconductor devices that may be used to produce an output pulse.

### Example 5: Experimental Results of High-Frequency, Bipolar Pulses For Electroporation of Cells

A chemical reaction technique was performed to fabricate parallel silver electrodes on glass microscope slides with 100 µm spacing. Briefly, a commercially available mirroring kit was used to deposit pure silver onto the microscope slides (Angel Gilding Stained Glass Ltd, Oak Park, IL). A negative thin film photoresist (#146DFR-4, MG Chemicals, Surrey, British Colombia, Canada) was laid on top of the slide and passed through an office laminator (#4, HeatSeal H212, General Binding Corporation, Lincolnshire, IL). A photomask printed at 20k DPI on a transparent film (Output City, Cad / Art Services Inc, Bandon, OR) was placed ink side down onto the photoresist, and slides were exposed to UV light for 45 seconds. After exposure, the slides were placed in a 200 mL bath containing a 10:1 DI water to negative photo developer (#4170-500ML, MG Chemicals, Surrey, British Colombia, Canada). The slides were placed in a beaker containing DI water to stop the development process and gently dried using pressurized air. Electrode structures on the microscope slides were fabricated by removing all silver not covered by the patterned photoresist. A two part silver remover was included in the mirroring kit used to deposit the silver. The photoresist was then removed by placing the slide in a bath of acetone.

Microfluidic channels were fabricated using the patterned photoresist on a microscope slide that had not undergone the silvering process. Liquid phase polydimethylsiloxane (PDMS) in a 10:1 ratio of monomers to curing agent (Sylgrad 184, Dow Corning, USA) was degassed under vacuum prior to being poured onto the photoresist master and cured for 1 hour at 100° C. After removing the cured PDMS from the mold, fluidic connections to the channels were punched in the devices using 1.5 mm core borers (Harris Uni-Core, Ted Pella Inc., Redding, CA). The PDMS mold was then bonded over the glass slides containing the patterned electrodes by treating with air plasma for 2 minutes in a PDC-001 plasma cleaner (Harrick Plasma, Ithaca, New York).

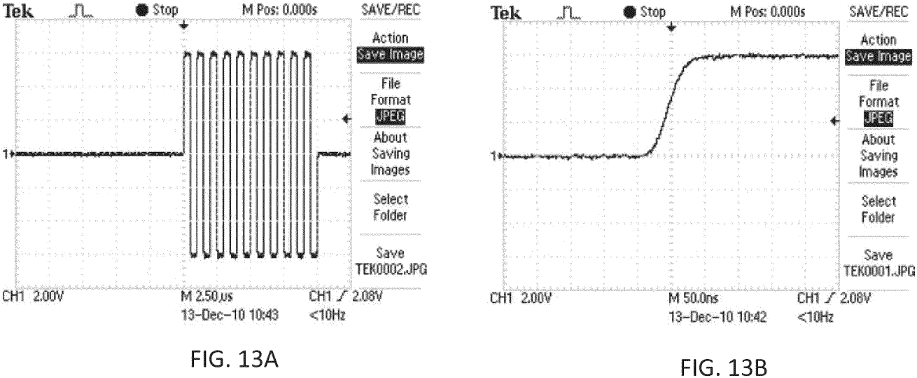

High voltage electrical wires were taped to the glass slide with exposed wire placed in direct contact with the electrical pads. A drop of high purity silver paint (Structure Probe Inc., West Chester, PA) was placed on the pad and allowed to dry for one hour creating a solid electrical connection. A drop of 5 minute epoxy (Devcon, Danvers, MA), used to secure the electrical connections, was placed on top of each electrode pad and allowed to cure for 24 hours. Pulses were delivered across the electrodes as described in EXAMPLE 4 prior to the amplification stage. No amplification was needed as the gap between the electrodes was only 100 µm. Therefore, the output signal of a function generator (GFG-3015, GW Instek, Taipei, Taiwan) +/-10 V can be used to generate an electric field capable of inducing electroporation, as shown in FIGS. 13A-B.

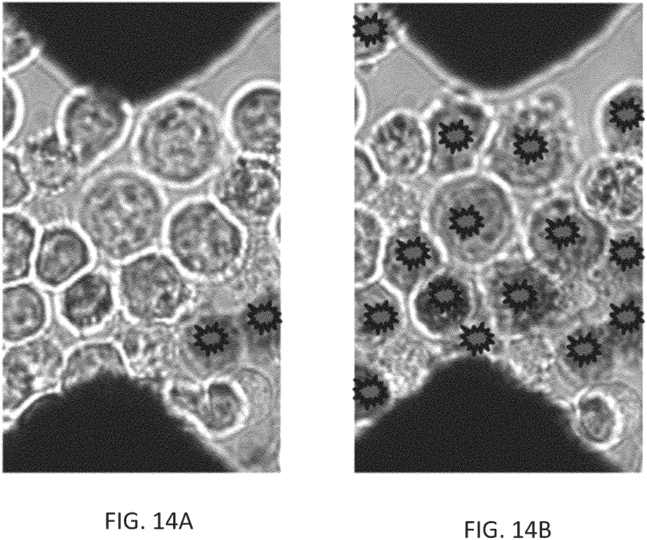

Following culture in DMEM-F12 (supplemented with 10% FBS and 1% penicillin streptomycin) MDA-MB-231 cells were resuspended in a PBS solution 1:1 with Trypan Blue (0.4 %). Trypan Blue is a determinant of cell membrane integrity, and stains electroporated cells blue, whereas non-electroporated cells remain transparent. Cells at a concentration of 10⁶/ml were injected into the microfluidic channel using a syringe. The function generator was triggered by the microcontroller to deliver 80, 50 kHz bursts with a width of 1 ms and an amplitude of 500 V/cm. Results shown in FIGS. 14A-B, which shows that 60% transfection efficiency was obtained when starting with cells that are 92% viable. This efficiency of reversible electroporation could be improved by either increasing the number of pulses or the burst width. Additionally, IRE could be performed by increasing the applied voltage.

### Example 6: Alternate Waveforms for Performing High-Frequency Electroporation

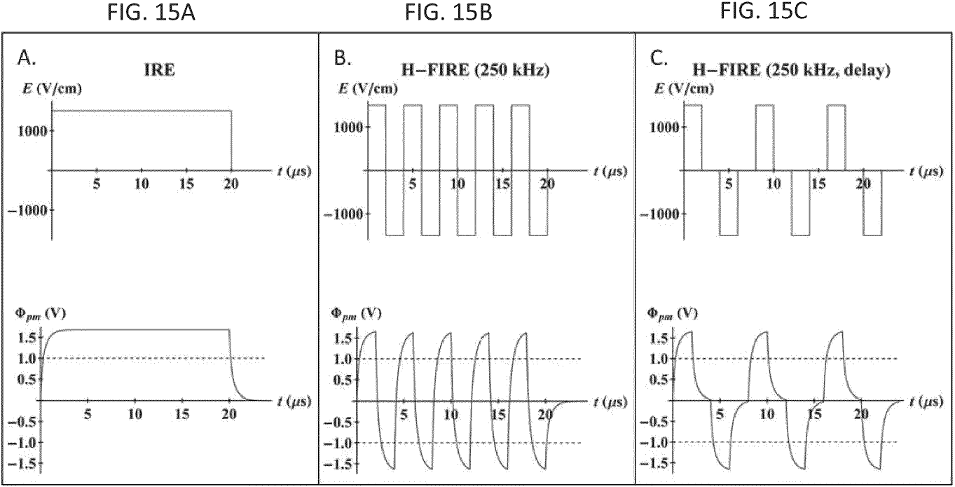

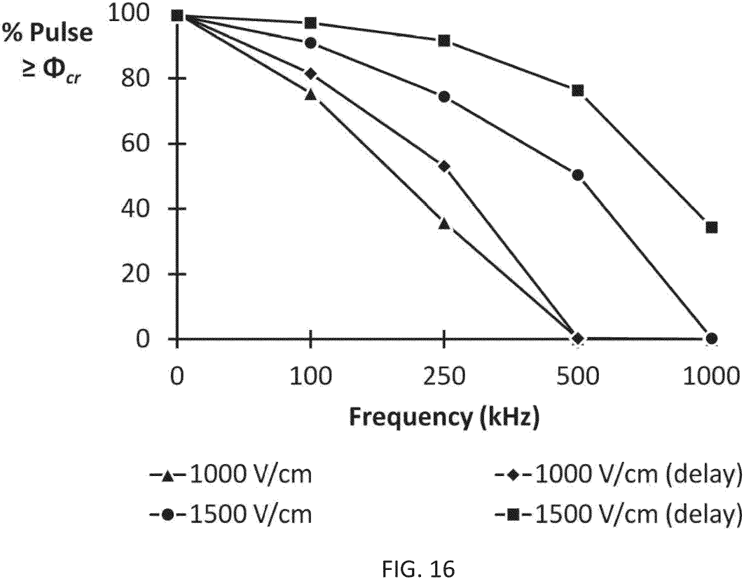

The analytical model for TMP described in the detailed description of the invention was utilized to investigate electroporation of a spherical cell subject to alternative waveforms. As mentioned, the critical TMP ( Φ_{cr} ) across the plasma membrane required to induce IRE is approximately 1 V. Belehradek, J., S. Orlowski, L.H. Ramirez, G. Pron, B. Poddevin, and L.M. Mir, Electropermeabilization of Cells in Tissues Assessed by the Qualitative and Quantitative Electroloading of Bleomycin. Biochimica Et Biophysica Acta-Biomembranes, 1994. 1190(1): p. 155-163. This threshold is illustrated in FIGS. 15A-C by the dashed, horizontal line on the TMP profiles. Characteristic waveforms of IRE with unipolar pulses and high-frequency IRE with the corresponding TMP development across the plasma membrane (Φₚₘ). All results are presented at the cell pole (θ = 0) to show the maximum TMP around the cell. Further, results are only shown for TMP across the plasma membrane, as the TMP across the nuclear envelope never approached the permeabilizing threshold. For an electric field of 1500 V/cm, results indicate that a unipolar pulse (FIG. 15A), a 250 kHz bipolar burst (FIG. 15B), and 250 kHz bipolar burst that includes delays between the pulses (FIG. 15C) are all capable of inducing IRE. However, the time above the threshold TMP varies between the different cases. The 1500 V/cm unipolar pulse causes the TMP to rise above the critical threshold for IRE (1 V, dashed line). The 1500 V/cm bipolar burst without a delay and with a delay causes the TMP to oscillate around the same critical threshold. This is investigated further in FIG. 16 for center frequencies of 0, 100, 250, 500, and 1000 kHz, with the 0 kHz case representing the unipolar pulse, and electric fields of 1000 V/cm and 1500 V/cm. FIG. 16 provides a comparison of time above the critical threshold (Φ_{cr}) for IRE at various center frequencies. The burst width of the bipolar waveform that included delays was twice as long (40 µs) as the corresponding burst with no delays in order to generate an equivalent pulse on-time (20 µs). The amount of time that the TMP was above the critical value was normalized by the on-time and converted to a percentage. FIG. 16 illustrates that, for a given frequency, as the electric field is increased from 1000 V/cm to 1500 V/cm, the percentage of the burst above the critical TMP also increases. At 250 kHz, IRE is possible during all waveforms, but at 500 kHz, only the waveforms with amplitudes of 1500 V/cm are capable of inducing IRE. As the center frequency of the burst increases, the percentage of the burst above the critical TMP decreases. However, with the inclusion of delays between the pulses, this characteristic dispersion is shifted towards higher frequencies. At 1 MHz, only the 1500 V/cm waveform with delays can theoretically cause IRE.

The theoretical model of TMP suggests that IRE should be possible up to 1 MHz for an electric field of 1500 V/cm. Including a delay between the positive and negative pulses comprising the bipolar burst offers a therapeutic advantage in addition to protecting the MOSFETs in the pulse generation system (see EXAMPLE 4) from ringing. By not forcing a discharge of the TMP with an immediate reversal of polarity, the cell is allowed to return to the resting TMP according to its characteristic time constant. As a result, the TMP is maintained above the critical voltage required for IRE for a longer amount of time. This metric has been recognized as a potential indicator of treatment outcomes in electroporation based therapies with bipolar waveforms. Garcia, P.A., J.H. Rossmeisl, R.E. Neal, T.L. Ellis, J.D. Olson, N. Henao-Guerrero, J. Robertson, and R.V. Davalos, Intracranial Nonthermal Irreversible Electroporation: In Vivo Analysis. Journal of Membrane Biology, 2010. 236(1): p. 127-136.

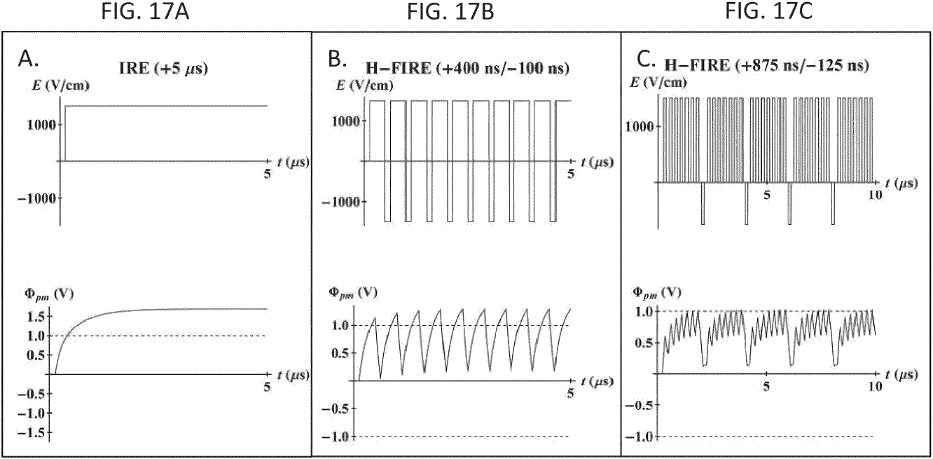

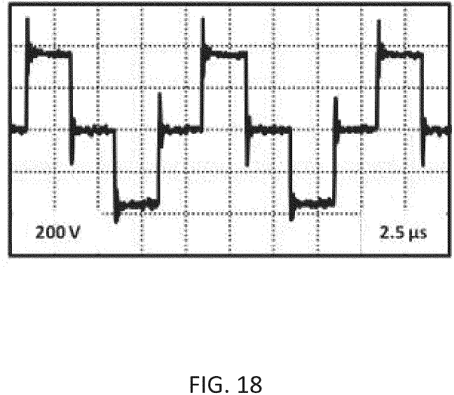

Other potential waveforms for performing high-frequency electroporation are shown in FIGS. 17A-C, which provide characteristic waveforms of IRE with unipolar pulses and high-frequency IRE with the corresponding TMP development across the plasma membrane (Φₚₘ). A unipolar pulse with an amplitude of 1500 V/cm is shown for comparison (FIG. 17A). A waveform without delays between polarity reversals (FIG. 17B) can maintain a positive TMP throughout the entire treatment if the duration of positive polarity is tuned to be slightly longer than the duration of negative polarity. Similarly, for a waveform that includes delays (FIG. 17C), a train of positive ultra-short pulses could be used to gradually increase the TMP up to the critical permeabilizing threshold, and a single ultra-short pulse of negative polarity could follow the train without causing the TMP to go negative. In both examples, the ultra-short negative going pulse is designed to maintain the predicted benefits of high-frequency electroporation. Namely, it is predicted that the negative going pulse will prevent action potential generation and still permit a degree of capacitive coupling across epithelial layers. FIG. 18 is a chart showing an exemplary output from an in vivo treatment of the brain with high-frequency, bipolar pulses, where the snapshot is taken within a single burst.

### Example 7: Experimental Results of High-Frequency IRE (H-FIRE) of Brain Tissue

H-FIRE was performed using a custom pulse generator as described in EXAMPLE 4 with minor modifications. An unregulated DC power supply was constructed to replace the both the high voltage sequencer and external capacitor in order to maintain a sufficient level of charge to deliver 20 A over a 100 µs burst. A center tapped 400 VA transformer (AS-4T320, Antek, Inc., North Arlington, NJ, USA) was rectified and smoothed by a capacitor bank to provide positive and negative power rails to the HV1000P and HV1000N, respectively. The voltage rails were controlled by adjusting the input voltage using a variable transformer, and the maximum output rating of the system was +/- 450 V. A delay equal to the duration of single polarity was included between the pulses in order to protect the MOSFETs from ringing. A unity gain inverting amplifier (AD844, Analog Devices, Norwood, MA, USA) was used to invert this signal and appropriately trigger the negative pulse generator. The outputs of the two monopolar pulse generators were terminated into a 50 Ω load in parallel with the electrodes. This load was used to maintain appropriate pulse characteristics and as a safety to ensure the system was never triggered without an attached load. For comparison, the IRE treatments were performed using the BTX ECM 830 electroporation system (Harvard Apparatus, Holliston, MA, USA).

All study procedures were conducted following Institutional Animal Care and Use Committee approval and performed in a GLP compliant facility. Four, Fischer 344 male rats weighing 200-240 g were anesthetized by intraperitoneal injection of 10 mg/kg xylazine and 60 mg/kg ketamine hydrochloride, and a surgical plane of anesthesia was assessed by loss of the tail pinch reflex. To monitor muscle contractions, a 3-axis accelerometer breakout board (ADXL335, Adafruit Industries, New York, NY, USA) with a sensing range of ± 3 g’s was sutured to the dorsum of each rat in the interscapular region at the cervicothoracic junction using 5-0 monocryl suture. Low-pass filter capacitors (0.1 µF) were included at the x, y, and z outputs of the accelerometer for noise reduction. The hair of the skull was clipped and aseptically prepared using povidone-iodine and alcohol solutions. Anesthetized rats were placed in a small animal stereotactic head frame (Model 1350 M, David Kopf Instruments, Tungisten, CA, USA). A routine lateral rostrotentorial surgical approach to the skull was made, and 6 mm by 3 mm rectangular parieto-occipital craniectomy defects were created in the right and left aspects of the skull of each rat using a high-speed electric drill. Custom electrodes were inserted into the center of the forelimb area of the sensorimotor cortex of each rat (coordinates relative to Bregma: 1 mm anterior, 2.5 mm lateral, 2 mm dorsoventral) and advanced to a depth of 2 mm beneath the surface of the exposed dura. The electrodes were fashioned by blunting stainless steel acupuncture needles (0.45 mm diameter, Kingli Medical Appliance Co., Wuxi, China) with high grade sandpaper. Exposure length was set to 1 mm by insulating the electrodes with miniature polyimide tubing (25 AWG, Small Parts, Seattle, WA, USA), and the edge-to-edge electrode spacing was set to 1 mm by molding the electrodes in liquid phase polydimethylsiloxane (PDMS) cured in a 10:1 ratio with Sylgard 184 (Dow Corning Corp., Midland, MI, USA) at 150° C. for 30 min.

Pulse parameters were chosen based on the results from the analytical and numerical models to ensure the greatest potential for non-thermal tissue ablation. Following electrode insertion, pulses were applied to the right and left cerebral hemispheres, resulting in two treatments per rat (Table 5).

TABLE 5

Pulse parameters of various treatment protocols

| Rat Number | Treatment | Hemisphere | Frequency (kHz) | Voltage (V) |
|---|---|---|---|---|
| 1 | IRE | Left | - | 100 |
| | H-FIRE | Right | 250 | 100 |
| 2 | IRE | Left | - | 200 |
| | H-FIRE | Right | 250 | 200 |
| 3 | H-FIRE | Left | 250 | 300 |
| | H-FIRE | Right | 250 | 400 |
| 4 | IRE | Left | - | 50 |
| | H-FIRE | Right | 500 | 400 |

H-FIRE experiments were performed using 180 bursts with a pulse on-time of 200 µs within each burst, and bursts were delivered at a rate of one per second. In Rat #1 and Rat #2, H-FIRE was applied at voltages of 100 V and 200 V, respectively, to the right hemisphere with a center frequency of 250 kHz (duration of single polarity equal to two microseconds). The left hemisphere of Rat #1 and Rat #2 were treated with 180 IRE pulses (200 µs duration) of equivalent energy. In Rat #3, H-FIRE was applied to the left and right hemispheres at voltages of 300 V and 400 V, respectively, with a frequency of 250 kHz. In Rat #4, H-FIRE was applied at a voltage of 400 V to the right hemisphere with a frequency of 500 kHz (duration of single polarity equal to one microsecond). The left hemisphere of Rat #4 was treated with 90 IRE pulses (200 µs) and an applied voltage of 50 V. This lower energy scenario was designed to compare H-FIRE treatment outcomes to traditional IRE protocols in the brain. Kotnik, T. and D. Miklavcic, Theoretical evaluation of voltage inducement on internal membranes of biological cells exposed to electric fields. Biophysical Journal, 2006. 90(2): p. 480-491.

Immediately following treatment, Rats #3 and #4 were subjected to MRI examinations of the brain while under general anesthesia. The MRI was performed with a 0.2 T MRI scanner using a dual phased array hand/wrist coil for RF signal transmission and reception. Sequence acquisition parameters were as follows: T1-weighted images were acquired using spin echo pulse sequence (TR = 200 ms, TE = 16 ms, FOV = 6 cm, matrix = 256 × 196, slice thickness = 2 mm), and T2-weighted images were acquired using a gradient echo pulse sequence (TR = 3000 ms, TE = 90 ms, FOV = 6 cm, matrix = 256 × 196, slice thickness = 3 mm). T1-weigthed images were obtained following intraperitoneal injection of 0.1 mmol/kg of gadopentetate dimeglumine (Magnevist, Berlex Laboratories, NJ, USA). In all rats, humane euthanasia was performed by cervical dislocation approximately 1 hr post-treatment, and the brain was removed and fixed intact in 10% neutral buffered formalin. Following fixation for 48 hours, an adult rat brain matrix slicer (Zivic Instruments, Pittsburg, PA) was used to obtain contiguous 2 mm coronal brain sections from each animal. Brain and sections were embedded routinely in paraffin, sectioned at 5 µm, and stained with hematoxylin and eosin (H&E).

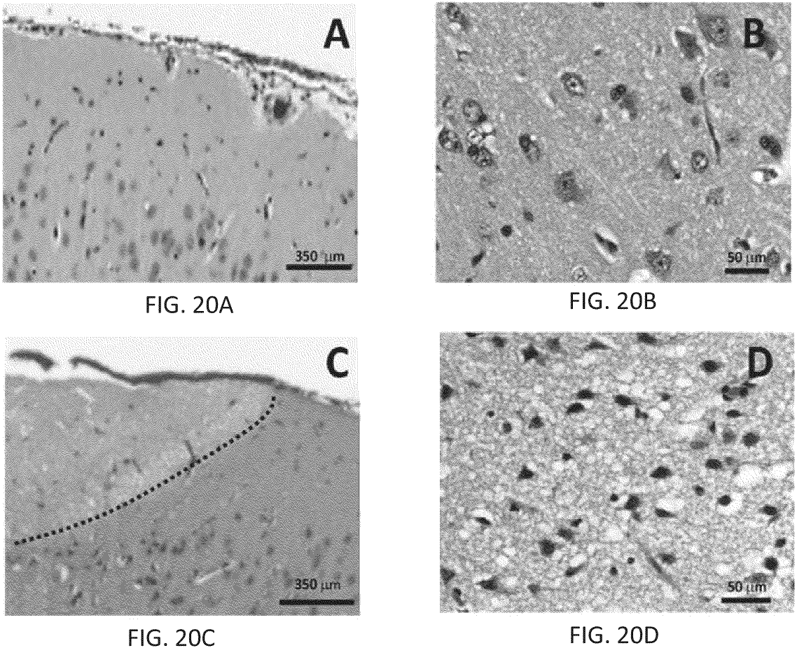

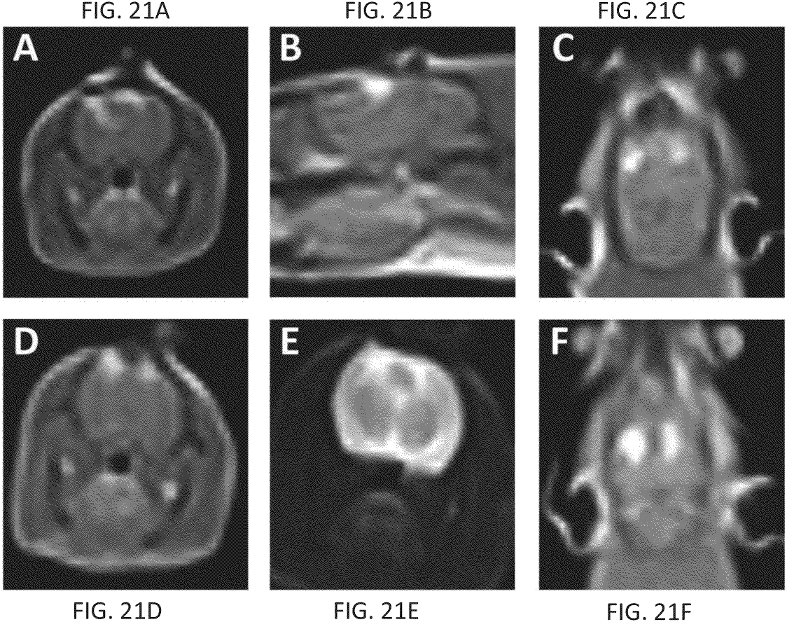

Treatments evaluated in this study produced ablative lesions in brain tissue, as evaluated with MRI examinations (FIGS. 21A-F) and pathologic preparations (FIGS. 20A-D). In Rats #3 and #4, the MRI characteristics of both H-FIRE and IRE lesions were similar. The MRI appearance of lesions in rat brain appeared as focal, ovoid to elliptical, T1 iso- to hypo-intense, uniformly and markedly contrast enhanced (FIGS. 21A, B, C, D, F) and T2 hyper-intense (FIG. 21E). In all panels, lesions appear as focal hyper-intense regions (white) compared to adjacent untreated cerebrocortical tissue (gray). Top Panels (A-C) obtained from Rat #3, in which both the left and right cerebral hemispheres were treated with high-frequency waveforms at 300 V/250 kHz and 400 V/250 kHz, respectively. Bottom Panels (D-F), Rat #4, which underwent high-frequency, bipolar pulses in the right cerebrum at 400 V/500 kHz, and conventional IRE with unipolar pulses at 50 V in the left cerebrum. Panels A and D, post-gadolinium T1-weighted MRI sequences in the axial plane. Panel B, post-gadolinium T1-weighted MRI sequences in the right parasagittal plane. Panels C and F, post-gadolinium T1-weighted MRI sequences in the dorsal plane. Panel D, T2-weighted MRI sequence in the transverse plane. In all panels, the right side of the brain is on the left side of the panel.

All lesions were well demarcated from adjacent, normal brain tissue and appeared similar in size. Compared to untreated brain (FIGS. 20A and B), histopathologic examination of brain sections from all treatments demonstrated clear areas of ablation indicated by pallor of the treated tissue that was sharply delineated from adjacent normal brain (FIG. 20C). H-FIRE and IRE lesions were predominantly characterized by areas of complete obliteration of cerebrocortical architecture by an eosinophilic, vacuolated amorphous debris (FIGS. 20C and D). In Rat #1, the H-FIRE ablation zone was confined to regions of elevated electric field surrounding the electrodes, whereas all other pulsing protocols resulted in ablation zones spanning the entire region between the electrodes. Cavitary cerebrocortical defects were induced with H-FIRE in Rat #1 and IRE in Rat #4. Variably sized regions of intraparenchymal hemorrhage were most pronounced immediately adjacent to and within electrode insertion tracks. The morphology of remnant neuronal and glial elements within H-FIRE ablated regions demonstrated features of both apoptosis and necrosis, including shrunken and hypereosinophilic cytoplasm, nuclear chromatin condensation, and nuclear pyknosis and karyolysis (FIG. 20D). Free glial and neuronal nuclei in various states of degeneration were scattered throughout ablation zones. Inflammation was not a significant feature of IRE or H-FIRE lesions at the time point brains were examined.

### Example 8: Elimination of Muscle Contractions During High-Frequency IRE (H-FIRE) of Brain Tissue

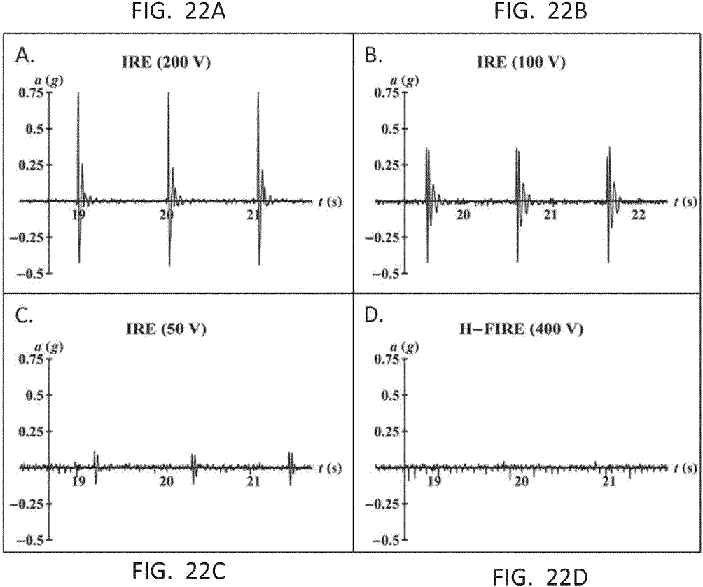

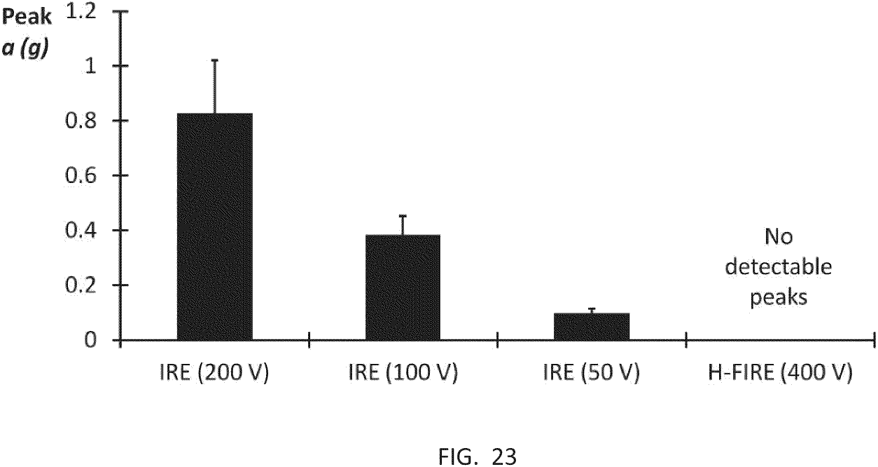

Muscle contractions were monitored throughout the procedure described in EXAMPLE 7 with the accelerometer located in the interscapular region at the cervicothoracic junction. All IRE pulsing protocols were associated with macroscopic muscular contractions of the cervicothoracic junction, which were also palpable to the neurosurgeon, while no visual or tactile evidence of muscular contraction was seen during any of the H-FIRE bursts. These results were quantitatively confirmed by the data recordings from the accelerometer (FIGS. 22A-D). Peak acceleration was determined during the first 90 bursts of the highest energy H-FIRE protocol (400 V/250 kHz) and the first 90 pulses of each IRE protocol (50 V, 100 V, 200 V). A one-way ANOVA was used to investigate the effects of each protocol on the ranks of peak acceleration at the cervicothoracic junction. In the event of a significant main effect, pairwise comparisons were completed using Tukey’s Honestly Significant Difference (HSD). All statistical analyses were conducted using JMP 7 (Cary, North Carolina, USA) with a significance level of p ≤ 0.05. Results indicate that, even in the highest energy H-FIRE protocol, there are no detectable peaks in acceleration above the inherent noise of the system. However, in all IRE protocols, peaks in acceleration associated with each pulse are detectable above the baseline noise. Further, pairwise comparisons between the various IRE protocols indicated that the mean peak acceleration during each treatment was energy dependent. Specifically, the mean peak acceleration decreased as the applied voltage decreased (FIG. 23).

### Example 9: Experimental Results of High-Frequency IRE (H-FIRE) of Liver Tissue

All study procedures were conducted following Institutional Animal Care and Use Committee approval and performed in a GLP compliant facility. Two, Fischer 344 male rats weighing 200-240 g were anesthetized by intraperitoneal injection of 10 mg/kg xylazine and 60 mg/kg ketamine hydrochloride, and a surgical plane of anesthesia was assessed by loss of the tail pinch reflex. A routine laparotomy surgical approach to the abdomen was made in order to expose the liver. Custom electrodes were inserted into the liver parenchyma and advanced to a depth of 2 mm beneath the surface. The electrodes were fashioned from steel pins (Dritz, 0.5 mm diameter), and the edge-to-edge electrode spacing was set to 1 mm by inserting the electrodes in a custom polycarbonate spacer.

In Rat #1, H-FIRE was applied at 1000 V/cm with 80 unipolar bursts at a center frequency of 2 MHz and, 50% duty cycle, and 50 µs burst width. In Rat #2 IRE was applied at an equivalent energy using 80 unipolar pulses with a duration of 50 µs and amplitude of 1000 V/cm. In all rats, humane euthanasia was performed by cervical dislocation approximately 1 hr post-treatment, and the liver was removed and fixed intact in 10% neutral buffered formalin. Following fixation for 48 hours, 5 mm sections from each animal were obtained and embedded routinely in paraffin, sectioned at 5 µm, and stained with hematoxylin and eosin (H&E).

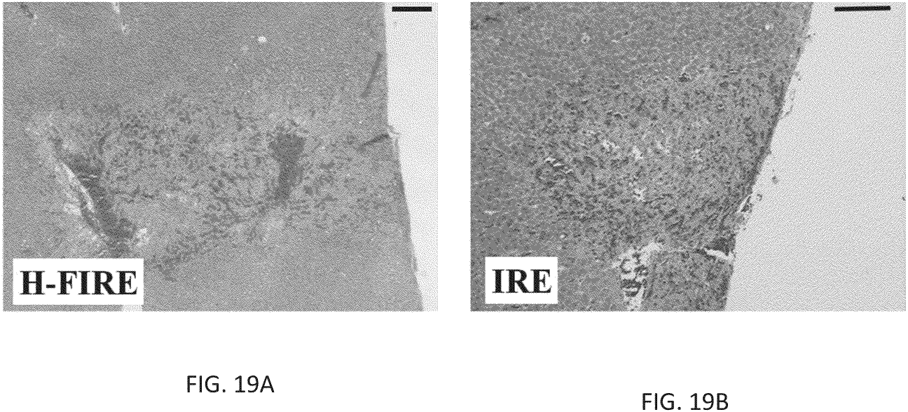

Histologically, in both treatments, there is evidence of necrosis and sinusoidal congestion (FIGS. 19A-B). Additionally, the lesions are well demarcated with cell scale resolution between treated and untreated tissue. Both of these features are common to IRE ablation of liver. No evidence of muscle contraction was observed visually. These results supplement those presented in EXAMPLE 7 and confirm that H-FIRE ablation can be achieved in multiple tissue types.

### Example 10: The Electric Field Distribution During High-Frequency Electroporation can be Approximated by the Laplace Equation

A 2D axisymmetric FEM representative of a slab of non-infiltrated fat adjacent to dry skin was simulated using COMSOL 4.2a (Burlington, MA). An energized and grounded electrode were modeled as infinite fins (0.5 mm diameter) separated 0.5 cm from the skin-fat interface, for a total spacing of 1 cm. The electric potential distribution within the tissue was obtained by transiently solving Equation 7 (see Example 1). Additionally, the homogeneous solution was solved according to the Laplace equation:

$$- ∇ ⋅ \left(∇ \text{Φ}\right) = 0$$

For the heterogeneous case, the dielectric properties of various tissues were chosen from data generated by Gabriel et al. available at (http://niremf.ifac.cnr.it/docs/dielectric/home.html). Gabriel, S., R.W. Lau, and C. Gabriel, The dielectric properties of biological tissues .2. Measurements in the frequency range 10 Hz to 20 GHz. Physics in Medicine and Biology, 1996. 41(11): p. 2251-2269. The data was interpolated in Mathematica 7 (Wolfram Research, Inc.) in order to estimate the dielectric properties at 1 kHz and 1 MHz. For the homogeneous case, the electric field distribution is independent of the dielectric properties. The energized and grounded electrodes were subtracted from the skin and fat subdomains, and treated purely as boundary conditions at 1000 V and 0 V, respectively.

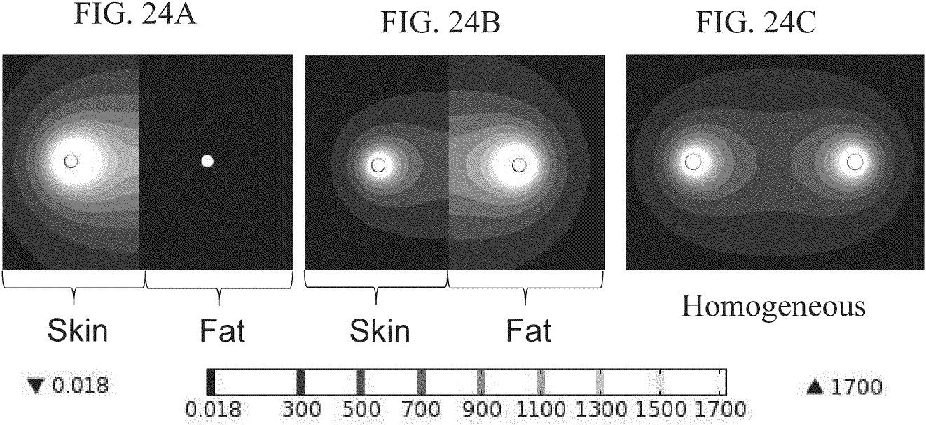

FIGS. 24A and B show the electric field distribution during a bipolar burst with the frequencies given in TABLE 6.

TABLE 6

Dielectric properties of skin and fat tissue at various frequencies

| Frequency | Property | Tissue | |
|---|---|---|---|
| | | Skin | Fat |
| 1 kHz | σ [S/m] | 0.000180 | 0.0246 |
| | εᵣ | 1170 | 20800 |
| 1 MHz | σ [S/m] | 0.0119 | 0.0267 |
| | εᵣ | 792 | 25 |

From the surface contour map, at 1 kHz, which is representative of a 500 µs traditional electroporation pulse, the electric field is highly non-uniform. A majority of the voltage drop occurs within the skin layer, and the fat layer remains untreated. However, at 1 MHz, which is representative of a 500 ns high-frequency electroporation pulse, the voltage drop is distributed more uniformly throughout the entire domain. As a result, both the skin and fat layers can be treated. Additionally, the electric field distribution at 1 MHz closely resembles that of the homogenous solution. Therefore, knowledge of dielectric properties and intricate geometrical arrangements of heterogeneous tissues can be neglected during treatment planning for high-frequency electroporation. This greatly reduces treatment planning protocols and produces more predictable outcomes.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. Where a range of values is provided in this specification, each value between the upper and lower limits of that range is also specifically disclosed. The upper and lower limits of these smaller ranges may independently be included or excluded in the range as well. As used in this specification, the singular forms “a,” “an,” and “the” include plural referents unless the context clearly dictates otherwise. It is intended that the specification and examples be considered as exemplary in nature and that variations that do not depart from the essence of the invention are intended to be within the scope of the invention. Further, the references cited in this disclosure are incorporated by reference herein in their entireties.

1-20. (canceled)

21. An electroporation system comprising:
at least one electrode; and
a voltage pulse generator coupled to the at least one electrode and configured to apply a plurality of electrical pulses to a target region comprising cells of tissue;
wherein the plurality of electrical pulses comprises bipolar or alternating polarity pulses, have a square waveform, and have a delay between successive pulses, wherein the length of each pulse and the delay between successive pulses are optimized to produce a first treatment zone and a second treatment zone;
wherein the voltage pulse generator is configured such that the plurality of electrical pulses is capable of non-thermal ablation in the first treatment zone; and
wherein the voltage pulse generator is configured such that in the second treatment zone, only selected cells, which are cancer cells, are killed by i) necrosis, ii) apoptosis, or iii) necrosis and apoptosis.

22. The system of claim 21, wherein the voltage pulse generator is configured such that:
within the first treatment zone cancer cells die quickly on the order of minutes; and
within the second treatment zone cancer cells die slower on the order of hours.

23. The system of claim 21, wherein the voltage pulse generator is configured to apply the plurality of electrical pulses such that cancer cells are affected by the plurality of electrical pulses and normal cells are not affected in the same way by the plurality of electrical pulses.

24. The system of claim 21, wherein the voltage pulse generator is configured to apply the plurality of electrical pulses such that cancer cells of a more malignant type are affected by the plurality of electrical pulses and cancer cells of a less aggressive type are not affected in the same way by the plurality of electrical pulses.

25. The system of claim 21, wherein the plurality of electrical pulses includes positive and negative pulses having different pulse widths or different amplitude.

26. The system of claim 21, wherein one or more pulses of the plurality of electrical pulses has a length of between 250 nanoseconds and 50 microseconds.

27. The system of claim 21, wherein the plurality of electrical pulses is applied at a frequency of between 1 kHz and 10 MHz.

28. The system of claim 21, wherein the delay between successive pulses is a fraction of the length of a pulse.

29. The system of claim 21, wherein one type of cell is affected by the plurality of the electrical pulses and another type of cell is not affected in the same way by the plurality of electrical pulses.

30. The system of claim 21, wherein the voltage pulse generator is capable of delivering the plurality of electrical pulses with a voltage in the range of from 0 to about 5,000 V.

31. An electroporation system comprising:
at least one electrode; and
a voltage pulse generator configured to be operatively coupled to the at least one electrode and configured to apply a plurality of electrical pulses to a target region comprising cells of tissue;
wherein a length of each pulse and a delay between successive pulses are optimized to produce a first treatment zone and a second treatment zone;
wherein the voltage pulse generator is configured such that the plurality of electrical pulses is capable of non-thermal ablation in the first treatment zone; and
wherein the voltage pulse generator is configured such that in the second treatment zone only selected cells, which are cancer cells, are killed by i) necrosis, ii) apoptosis, or iii) necrosis and apoptosis.

32. The system of claim 31, wherein the plurality of electrical pulses comprises bipolar or alternating polarity pulses.

33. The system of claim 31, wherein the plurality of electrical pulses have a square waveform.

34. The system of claim 31, wherein one or more pulses of the plurality of electrical pulses has a length between 250 nanoseconds and 50 microseconds.

35. The system of claim 31, wherein the first treatment zone comprises cancer cells and non-cancer cells which are killed by necrosis.

36. The system of claim 31, wherein the voltage pulse generator is configured to apply the plurality of electrical pulses such that cancer cells of a more malignant type are affected by the plurality of electrical pulses and cancer cells of a less aggressive type are not affected in the same way by the plurality of electrical pulses.

37. The system of claim 31, wherein the voltage pulse generator is capable of delivering the plurality of electrical pulses with a voltage in the range of from 0 to about 5,000 V.

38. The system of claim 31, wherein a length of each pulse is equivalent to a charging time of a cell membrane of the selected cells plus a discharge time of a nuclear membrane of the selected cells, while a delay between successive pulses is equivalent to the charging time of the cell membrane of the selected cells.

39. The system of claim 38, wherein
the charging time of the cell membrane of the selected cells and the discharge time of the nuclear membrane of the selected cells are determined through numerical modeling.

40. The system of claim 21, wherein the voltage pulse generator is configured to apply the plurality of electrical pulses in a manner such that the plurality of electrical pulses comprises an electric field waveform which is:
a rectangular pulse, ramp, decaying exponential, or sine wave; and
unipolar or bipolar.
